(19) United States
(12) Patent Application Publication
(10) Pub. No.: US 2024/0237898 A1
(43) Pub. Date: Jul. 18, 2024

(54) Endoscopy Based on Optical Coherence Tomography (OCT) and Convolutional Neural Networks (CNNs)

(71) Applicant: The Board of Regents of the University of Oklahoma, Norman, OK (US)

(72) Inventors: Qinggong TANG, Norman, OK (US); Chongle PAN, Norman, OK (US); Chen WANG, Norman, OK (US); Justin C. REYNOLDS, Norman, OK (US); Paul CALLE, Norman, OK (US); Sanjay G. PATEL, Oklahoma City, OK (US)

(21) Appl. No.: 18/428,985

(22) Filed: Jan. 31, 2024

Related U.S. Application Data
(63) Continuation-in-part of application No. 17/530,131, filed on Nov. 18, 2021.
(60) Provisional application No. 63/482,410, filed on Jan. 31, 2023.
(60) Provisional application No. 63/115,452, filed on Nov. 18, 2020.

(51) Int. Cl. A61B 5/00; A61B 17/34; G16H 20/40

(52) U.S. Cl. CPC A61B 5/0066; A61B 5/7264; A61B 17/3401; A61B 17/3478; G16H 20/40

(57) ABSTRACT

A method comprises: obtaining an endoscope; obtaining a needle; inserting the endoscope into the needle to obtain a system; inserting the system into an animal body; obtaining an image of a tissue or a space in the animal body using the endoscope and OCT; performing identification of the tissue or the space based on an OCT system; estimating a distance from the needle to the tissue or the space based on the identification and the OCT system; and performing a procedure with the needle and based on the identification and the distance.

### CROSS-REFERENCE TO RELATED APPLICATIONS

This claims priority to U.S. Prov. Patent App. No. 63/482,410 filed on Jan. 31, 2023 and is a continuation-in-part of U.S. patent application Ser. No. 17/530,131 filed on Nov. 18, 2021, which claims priority to U.S. Prov. Patent App. No. 63/115,452 filed on Nov. 18, 2020, all of which are incorporated by reference.

### STATEMENT REGARDING FEDERALLY SPONSORED RESEARCH OR DEVELOPMENT

This invention was made with government support under Contract Number DK133717 awarded by the National Institutes of Health (NIH). The government has certain rights in the invention.

### BACKGROUND

NBIs are procedures that require a minimally-invasive approach to gain access to tissue structures of interest. Each year, more than 30 million of these interventions are performed in the United States. Due to the lack of proper visual feedback guiding navigation, up to 33% of NBIs are associated with complications, incurring significant human and economic costs.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 is a schematic diagram of a forward-view endoscopic OCT system.

FIG. 2 is a schematic diagram of data acquisition and processing.

FIG. 3A is a diagram of an endoscope scanning a kidney sample.

FIG. 3B shows 3D OCT images, 2D cross-sectional images, and histology results of a renal cortex.

FIG. 3C shows 3D OCT images, 2D cross-sectional images, and histology results of a renal medulla.

FIG. 3D shows 3D OCT images, 2D cross-sectional images, and histology results of a renal calyx.

FIG. 4 is a table showing the average validation accuracies and their standard errors for the PT or RI model architectures after hyperparameter optimization.

FIG. 5 is a table showing the test accuracy of the best-performing model in each of the 10 testing folds.

FIG. 6A is a graph showing the ROC curve of the prediction results from kidney number 5.

FIG. 6B is a graph showing the ROC curve of the prediction results from kidney number 10.

FIG. 6C is a graph showing ROC curves for the three tissue types.

FIG. 7 is a table showing the average confusion matrix for the 10 kidneys in the 10-fold cross-testing with a score threshold of 0.333 and the average recall and precision for each type of tissue.

FIG. 8A shows class activation heatmaps for RI ResNet50 for three representative images in each tissue type.

FIG. 8B shows class activation heatmaps for PT ResNet50 for three representative images in each tissue type.

FIG. 9A shows images of a blood vessel in front of a needle tip as detected by Doppler OCT.

FIG. 9B shows images of manually-labeled regions of blood vessels serving as a ground truth.

FIG. 9C shows images demonstrating the predicted vessel regions by nnU-net.

FIG. 9D shows images of superimposed OCT Doppler images with manually-labeled blood vessel regions.

FIG. 9E shows images of superimposed OCT Doppler images with predicted blood vessel regions by nnU-net.

FIG. 10 is a flowchart illustrating a method for endoscopic guidance using neural networks according to one non-limiting embodiment of the present disclosure.

FIG. 11 is a schematic diagram of an apparatus according to one non-limiting embodiment of the present disclosure.

FIG. 12 is a schematic of an endoscopic OCT system for Veress needle guidance.

FIG. 13 is a flow diagram of data acquisition and processing.

FIG. 14 is a flowchart of a process of nested cross-validation, cross-testing, and 8-fold cross-validation.

FIG. 15 shows examples of 2D OCT results of the three different tissues and abdominal space.

FIG. 16 is a table showing the average nested 7-fold cross-validation accuracies for tissue-layer classification.

FIG. 17 is a table of the 8-fold cross-testing accuracies on the testing set in each testing fold.

FIG. 18 is a graph of aggregated ROC across all 8 testing folds using the Xception model.

FIG. 19 are heatmaps of tissue classification activation obtained from sample 1.

FIG. 20 is a table showing the average nested cross-validation MAPE with standard error in each nested cross-validation fold.

FIG. 21 is a table of MAPE with SE and MAE with SE during 8-fold cross-testing.

FIG. 22A shows between the manually labeled results (ground truth value) and predicted results.

FIG. 22B shows violin plots from sample one.

FIG. 23A is a schematic diagram of the experiment using the endoscopic OCT system.

FIG. 23B shows cross-sectional 2D OCT image examples of fat, interspinous ligament, ligamentum flavum, epidural space and spinal cord.

FIG. 24 is a table of average accuracies and standard error based on the practical tissue layer sequence during puncture for cross-validation.

FIG. 25 is a table further showing the test accuracy of the best-performing model (ResNet50) in each of the 8 testing folds.

FIG. 26A shows class activation heatmaps of ResNet50 models for representative images to show the salient features used for classification.

FIG. 26B shows some images from a video that can be found in the Github repository.

FIG. 27 is a table showing the mean and standard error of the cross-validation MAPE for ResNet50.

FIG. 28A shows examples of OCT images with different distances between needle tip and tissue.

FIG. 28B shows violin plots of the distribution of the errors from the Inception model during the seventh testing fold.

FIG. 29 is a schematic diagram of the forward-view OCT endoscope.

FIG. 30 is a diagram showing the data acquisition process.

FIG. 31 is a flowchart illustrating a method for endoscopy based on OCT and CNNs according to one non-limiting embodiment of the present disclosure.

### DETAILED DESCRIPTION

It should be understood at the outset that, although an illustrative implementation of one or more embodiments are provided below, the disclosed systems and/or methods may be implemented using any number of techniques, whether currently known or in existence. The disclosure should in no way be limited to the illustrative implementations, drawings, and techniques illustrated below, including the exemplary designs and implementations illustrated and described herein, but may be modified within the scope of the appended claims along with their full scope of equivalents.

Before further describing various embodiments of the apparatus, component parts, and methods of the present disclosure in more detail by way of exemplary description, examples, and results, it is to be understood that the embodiments of the present disclosure are not limited in application to the details of apparatus, component parts, and methods as set forth in the following description. The embodiments of the apparatus, component parts, and methods of the present disclosure are capable of being practiced or carried out in various ways not explicitly described herein. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. While the apparatus, component parts, and methods of the present disclosure have been described in terms of particular embodiments, it will be apparent to those of skill in the art that variations may be applied to the apparatus, component parts, and/or methods and in the steps or in the sequence of steps of the method described herein without departing from the concept, spirit, and scope of the inventive concepts as described herein. All such similar substitutes and modifications apparent to those having ordinary skill in the art are deemed to be within the spirit and scope of the inventive concepts as disclosed herein.

All patents, published patent applications, and non-patent publications referenced or mentioned in any portion of the present specification are indicative of the level of skill of those skilled in the art to which the present disclosure pertains, and are hereby expressly incorporated by reference in their entirety to the same extent as if the contents of each individual patent or publication was specifically and individually incorporated herein.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms and phrases, unless otherwise indicated, shall be understood to have the following meanings: The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The phrase “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the apparatus, composition, or the methods or the variation that exists among the objects, or study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately”, where used herein when referring to a measurable value such as an amount, percentage, temporal duration, and the like, is meant to encompass, for example, variations of +20% or +10%, or +5%, or +1%, or +0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. The range 100 units to 2000 units therefore refers to and includes all values or ranges of values of the units, and fractions of the values of the units and integers within said range, including for example, but not limited to 100 units to 1000 units, 100 units to 500 units, 200 units to 1000 units, 300 units to 1500 units, 400 units to 2000 units, 500 units to 2000 units, 500 units to 1000 units, 250 units to 1750 units, 250 units to 1200 units, 750 units to 2000 units, 150 units to 1500 units, 100 units to 1250 units, and 800 units to 1200 units. Any two values within the range of about 100 units to about 2000 units therefore can be used to set the lower and upper boundaries of a range in accordance with the embodiments of the present disclosure. More particularly, a range of 10-12 units includes, for example, 10, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, and 12.0, and all values or ranges of values of the units, and fractions of the values of the units and integers within said range, and ranges which combine the values of the boundaries of different ranges within the series, e.g., 10.1 to 11.5.

The following abbreviations apply:
ASIC: application-specific integrated circuit
AUC: area under the ROC curve
BD: balanced detector
BE: Barrett's esophagus
CCD: charge-coupled device
CNN: convolutional neural network
CPU: central processing unit
CT: computed tomography
DAQ: data acquisition
dB: decibel(s)
DOCT: Doppler optical coherence tomography
DSP: digital signal processor
EO: electrical-to-optical
FC: fiber coupler
FOV: field of view
FN: false negative
FP: false positive
FPGA: field-programmable gate array
GI: gastrointestinal
GPU: graphics processing unit
GRAD-CAM: gradient-weighted class activation mapping
GRIN: gradient-index
GSM: galvanometer scanning mirror
H&E: hematoxylin and eosin
kHz: kilohertz
LOR: loss of resistance
MAE: mean absolute error
MAPE: mean absolute percentage error
MEMS: microelectromechanical systems
mIoU: mean intersection-over-union
mm: millimeter(s)
MRI: magnetic resonance imaging
ms: millisecond(s)
mW: milliwatt(s)
MZI: Mach-Zehnder interferometer
NBI: needle-based intervention
NIH: National Institutes of Health
nm: nanometer(s)
OCT: optical coherence tomography
OE: optical-to-electrical
PC: polarization controller
PCN: percutaneous nephrostomy
PCNL: percutaneous nephrolithotomy
PDPH: post-dural puncture headache
PS-OCT: polarization-sensitive OCT
PT: pre-trained
RAM: random-access memory
ResNet: residual neural network
RF: radio frequency
RI: randomly-initialized
ROC: receiver operating characteristic
ROM: read-only memory
RX: receiver unit
SE: standard error
SGD: stochastic gradient descent
SRAM: static RAM
SS-OCT: swept-source OCT
TCAM: ternary content-addressable memory
TN: true negative
TP: true positive
TX: transmitter unit
2D: two-dimensional
3D: three-dimensional
μm: micrometer(s)
°: degree(s).

### I. Endoscopic Guidance Using Neural Networks

PCN was first described in 1955 as a minimally-invasive, x-ray guided procedure in patients with hydronephrosis. PCN needle placement has since become a valuable medical resource for minimally-invasive access to the renal collecting system for drainage, urine diversion, the first step for PCNL surgery, and other therapeutic intervention, especially when the transurethral access of surgical tools into the urological system is difficult or impossible. Despite being a common urological procedure, it remains technically challenging to insert the PCN needle correctly in the right place. During PCN, a needle penetrates the cortex and medulla of the kidney to reach the renal pelvis. Conventional imaging modalities have been used in PCN puncture. Ultrasound technique, as a commonly used medical diagnostic imaging method, has been utilized in PCN surgery for decades. Additionally, fluoroscopy and CT are also employed in PCN guidance, and sometimes they are used with ultrasonography simultaneously. However, due to the limited spatial resolution, these standard imaging modalities have proven to be inadequate for accurately locating the needle tip position. The failure rate of PCN needle placement is up to 18%, especially in non-dilated systems or for complex stone diseases. Failure of inserting the needle into the targeted location in the kidney through a suitable route might result in severe complications. Moreover, fluoroscopy has no soft tissue contrast and, therefore, cannot differentiate critical tissues, such as blood vessels, which are important to avoid during the needle insertion. Rupture of renal blood vessels by needle penetrations can cause bleeding. Temporary bleeding after PCN placement occurs in ˜95% of cases. Retroperitoneal hematomas have been found in 13% of cases. When PCNL is followed, hemorrhage requiring transfusion increases to 12-14% of the patients. Additionally, needle punctures during PCN can lead to infectious complications such as fever or sepsis, thoracic complications like pneumothorax or hydrothorax, and other complications like urine leak or rupture of the pelvicalyceal system.

Therefore, the selection of position and route of the puncture is important in PCN needle placement. It is recommended to insert the needle into the renal calyx through calyx papilla because fewer blood vessels are distributed on this route, leading to a lower possibility of vascular injury. Nevertheless, it is always difficult, even for experienced urologists, to precisely identify this preferred inserting route in complicated clinical settings. If PCN puncture is executed multiple times, the likelihood of renal injury increases and the operational time lengthens, resulting in higher risks of complications.

To better guide PCN needle placement, substantive research work has been done to improve the current guidance practice. Ultrasound with technical improvements in many aspects has been utilized. For instance, contrast-enhanced ultrasound has been proved to be a potential modality in the guidance of PCN puncture. Tracked ultrasonography snapshots are also a promising method to improve the needle guidance. In order to resolve bleeding during needle puncture, combined B-mode and color Doppler ultrasonography has been applied in PCN surgeries and it provides promising efficiency in decreasing major hemorrhage incidence. Moreover, developments in other techniques such as cone-beam CT, retrograde ureteroscopy, and magnetic field-based navigation devices have been utilized to improve the guidance of PCN needle access. On the other hand, an endoscope can be assembled within a PCN needle and effectively improve the precision of PCN needle punctures, resulting in lower risks of complications and fewer times of insertions. However, most of the conventional endoscopic techniques involving CCD cameras can only provide 2D information and cannot detect subsurface tissue before the needle tip damages it. Thus, there is a critical need to develop new guiding techniques which have depth-resolved capability for PCN.

OCT is a well-established, non-invasive biomedical imaging modality which can image subsurface tissue with the penetration depth of several millimeters. By obtaining and processing the coherent infrared light backscattered from the reference arm and sample arm, OCT can provide 2D cross-sectional images with high axial resolution (˜10 μm), which is 10-100 times higher than conventional medical imaging modalities (e.g., CT and MRI). Owing to the high speed of laser scanning and data processing, 3D images of the detected sample formed by numerous cross-sectional images can be obtained in real time. Because of the differences in tissue structures among the renal cortex, medulla, and calyx, OCT has the potential to distinguish different renal tissue types. Due to a 1-2 mm penetration limitation in biological tissues, studies in kidneys using OCT have mainly focused on the renal cortex. OCT can be integrated with fiber-optic catheters and endoscopes for internal imaging applications. For example, endoscopic OCT imaging has been demonstrated in the human GI tract to detect BE, dysplasia, and colon cancer. A portable, hand-held forward-imaging endoscopic OCT needle device has been developed for real-time epidural anesthesia surgery guidance. This endoscopic OCT setup holds the promise in PCN guidance.

Given the enormous accumulation of images and inter- and intra-observer variation from subjective interpretation, computer-aided automatic methods have been utilized to accurately and efficiently classify these data. In automated OCT image analysis, CNNs have been demonstrated to be promising in various applications, such as hemorrhage detection of retina versus cerebrum and tumor tissue segmentation.

Embodiments provide endoscopic guidance using neural networks. In an embodiment, a forward-view OCT endoscopic system images kidney tissues lying ahead of a PCN needle during PCN surgery to access the renal calyx. This may be done to remove kidney stones. In another embodiment, similar imaging is used for percutaneous renal biopsies, urine drainage, urine diversion, and other therapeutic interventions in the kidney. The embodiments provide for neural networks, for instance CNNs, which can distinguish types of renal tissue and other components. The types of renal tissue include the cortex, medulla, and calyx. Other components include blood vessels and diseased renal tissues. By distinguishing the types of renal tissue and other components, the embodiments provide for injection of a needle into the desired tissue and provide for avoidance of undesired components.

In an experiment, images of the renal cortex, medulla, and calyx were obtained from ten porcine kidneys using the OCT endoscope system. The tissue types were clearly distinguished due to the morphological and tissue differences from the OCT endoscopic images. To further improve the guidance efficacy and reduce the learning burden of the clinical doctors, a deep-learning-based, computer-aided diagnosis platform automatically classified the OCT images by the renal tissue types. A tissue type classifier was developed using the ResNet34, ResNet50, and MobileNetv2 CNN architectures. Nested cross-validation and testing were used for model selection and performance benchmarking to account for the large biological variability among kidneys through uncertainty quantification. The predictions from the CNNs were interpreted to identify the important regions in the representative OCT images used by the CNNs for the classification.

ResNet50-based CNN models achieved an average classification accuracy of 82.6%+3.0%. The classification precisions were 79%+4% for cortex, 85%+6% for medulla, and 91%+5% for calyx, and the classification recalls were 68%+11% for cortex, 91%+4% for medulla, and 89%+3% for calyx. Interpretation of the CNN predictions showed the discriminative characteristics in the OCT images of the three renal tissue types. The results validated the technical feasibility of using this novel imaging platform to automatically recognize the images of renal tissue structures ahead of the PCN needle in PCN surgery.

### A. System

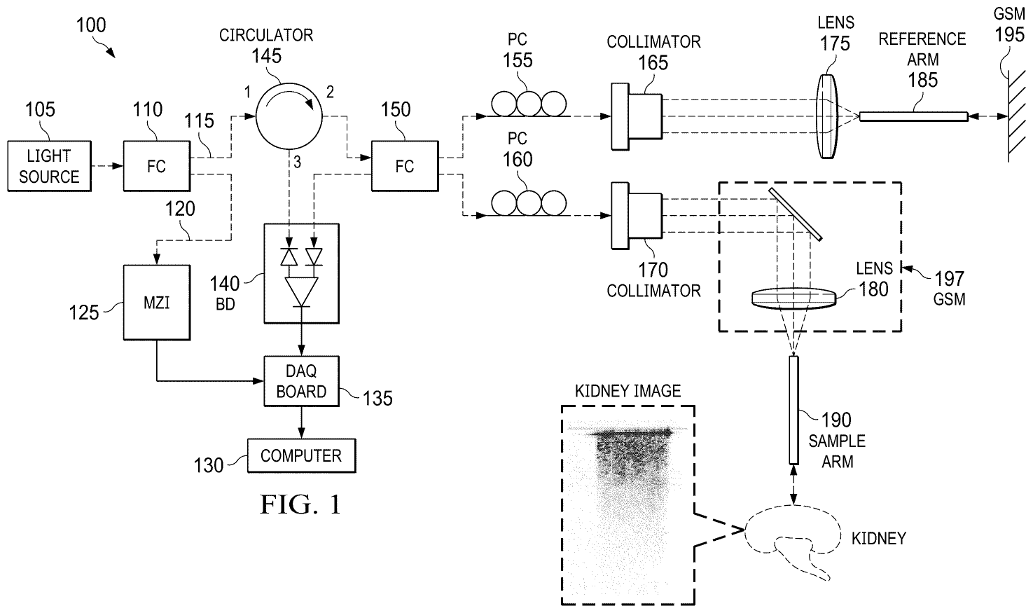

FIG. 1 is a schematic diagram of a forward-view endoscopic OCT system 100. The forward-view endoscopic OCT system 100 is based on an SS-OCT system and comprises a light source 105, an FC 110, a top path 115, a bottom path 120, an MZI 125, a computer 130, a DAQ board 135, a BD 140, a circulator 145, an FC 150, a PC 155, a PC 160, a collimator 165, a collimator 170, a lens 175, a lens 180, a reference arm 185, a sample arm 190, a GSM 195, and a GSM 197. The lenses 175, 180 are GRIN rod lenses with a 1.3 mm diameter. The reference arm 185 and the sample arm 190 form a Michelson interferometer. The forward-view endoscopic OCT system 100 could have an inner diameter of 0.1-8.0 mm, a length of 5-400 mm, and a view angle of 0-50°. With a protective steel tubing, the forward-view endoscopic OCT system 100 has an outer diameter of about 0.35-10.00 mm.

The light source 105 generates a laser beam with a center wavelength of 1300 nm and a bandwidth of 100 nm. The wavelength-swept frequency (A-scan) rate is 200 kHz with an ˜25 mW output power. The FC 110 splits the laser beam into a first beam with 97% of the whole laser power on the top path 115 and a second beam with 3% of the whole laser power on the bottom path 120. The second beam delivers into the MZI 125 for the MZI 125 to generate a frequency clock signal. The frequency clock signal triggers an OCT sampling procedure and passes to the DAQ board 135. The first beam passes to the circulator 145, which runs only in one direction. Therefore, the light entering port 1 only emits from port 2, and then it evenly splits towards the reference arm 185 and the sample arm 190. Backscattered light from both the reference arm 185 and the sample arm 190 form interference fringes at the FC 150 and transmit to the BD 140. The interference fringes from different depths received by the BD 140 are encoded with different frequencies. The BD 140 transmits an output signal to the DAQ board 135 and the computer 130 for processing. Cross-sectional information can be obtained through a Fourier transform of the interference fringes.

In the experiment, the lenses 175, 180 were stabilized in front of GSMs 195, 197. The proximal GRIN lens entrance of the endoscope was placed close to the focal plane of the objective lens. The GRIN lens can preserve the spatial relationship between the entrance and the output (distal end) and further to the sample. Therefore, one or two directional scanning can be readily performed on the proximal GRIN lens surface to create 2D or 3D images. In addition, the same GRIN rod lens was put in the light path of the reference arm 185 for the purpose of compensating light dispersion and expanding the length of the reference arm 185. The PCs 155, 160 decreased background noise. The forward-view endoscopic OCT system 100 had an axial resolution of ˜ 11 μm and lateral resolution of ˜20 μm in tissue. The lateral imaging FOV was around 1.25 mm. The sensitivity of the forward-view endoscopic OCT system 100 was optimized to 92 dB and calculated using a silver mirror with a calibrated attenuator.

### B. Data Acquisition

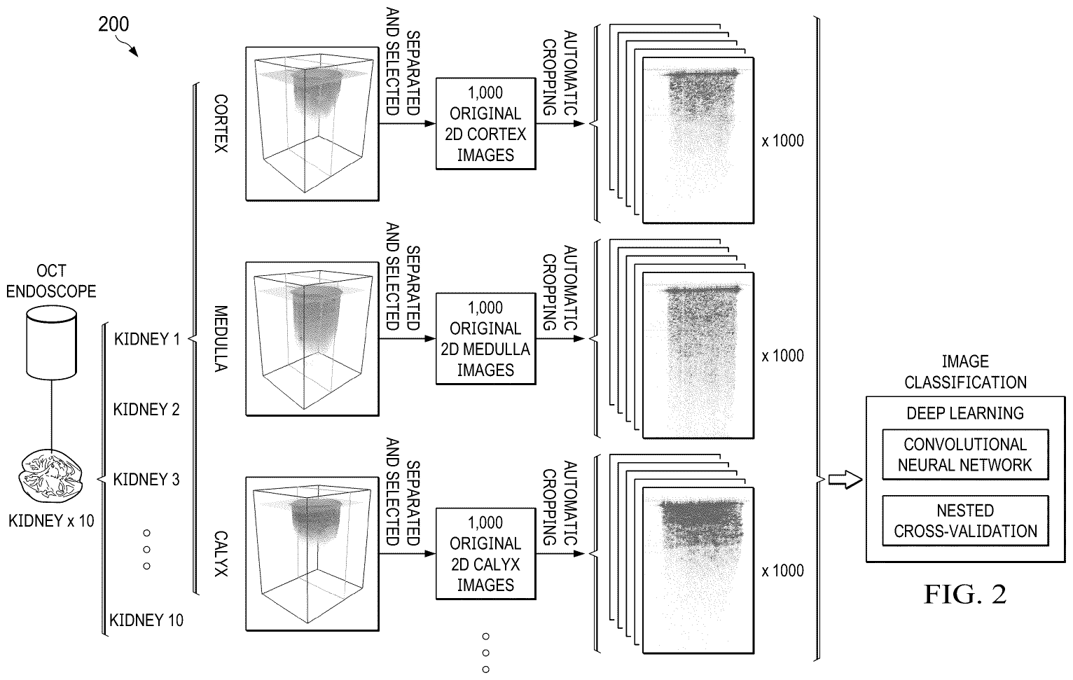

Ten fresh porcine kidneys were obtained from a local slaughterhouse. The cortex, medulla, and calyx of the porcine kidneys were exposed and imaged in the experiment. Renal tissue types can be identified from the anatomic appearance. The forward-view endoscopic OCT system 100 was placed against different renal tissues for image acquisition. To mimic a clinical situation, some force was applied while imaging the ex-vivo kidney tissues to generate tissue compression. 3D images of 320×320×480 pixels on X, Y and Z axes (Z presents the depth direction) were obtained with the pixel size of 6.25 μm on all three axes. Therefore, the size of the original 3D images is 2.00 mm×2.00 mm×3.00 mm. For every kidney sample, at least 30 original 3D OCT images were obtained for each tissue type, and each 3D tissue scanning took no more than 2 seconds. Afterwards, the original 3D images were separated to 2D cross-sectional images as shown in FIG. 2. FIG. 2 is a schematic diagram 200 of data acquisition and processing.

Since the GRIN lens is cylindrical, the 3D OCT images obtained were also in the cylindrical shape. Therefore, not all of the 2D cross-sectional images contained the same structural signal of the kidney. Only the 2D images with sufficient tissue structural information (cross-sectional images close to the center of the 3D cylindrical structures) were subsequently selected and utilized for the image preprocessing. At the end of imaging, tissues of cortex, medulla, and calyx of the porcine kidneys were excised and processed for histology to compare with corresponding OCT results. The tissues were fixed with 10% formalin, embedded in paraffin, sectioned (4 μm thick) and stained with H&E for histological analysis. Images were taken by Keyence Microscope BZ-X800.

Although the three tissue types showed different imaging features for visual recognition, it will take time and expertise for doctors to differentiate them during surgeries. In order to improve the efficiency, we developed deep learning methods for automatic tissue classification based on the imaging data. In total, ten porcine kidneys were imaged in this study. For each kidney, 1,000 2D cross-sectional images were obtained for each cortex, medulla, and calyx. For the purpose of convenient analysis and increasing the speed of deep-learning process of the OCT images, a custom MATLAB algorithm was designed to recognize the surface of the kidney tissue on the 2D cross-sectional images. The algorithm automatically cropped the images from the size of 320×480 to 235×301. Therefore, all the 2D cross-sectional images have the same dimensions and cover the same FOV before deep-learning processing.

### C. CNN Training

A CNN was used to classify the images of the renal cortex, medulla, and calyx. ResNet34, ResNet50, and MobileNetv2 were tested using Tensorflow 2.3 in open-ce version 0.1.

Pre-trained ResNet50 and MobileNetv2 models on the ImageNet dataset were imported. The output layer of the models was changed to one containing 3 softmax output neurons for cortex, medulla, and calyx. The input images were preprocessed by resizing to the 224×224 resolution, replicating the input channel to 3 channels, and scaling the pixel intensities to [−1, 1]. Model fine-tuning was conducted in two stages. First, the output layer was trained with all the other layers frozen. The optimizer, SGD, was used with a learning rate of 0.2, a momentum of 0.3, and a decay of 0.01. Then, the entire model was unfrozen and trained. The SGD with Nesterov momentum optimizer was used with a learning rate of 0.01, a momentum of 0.9, and a decay of 0.001. Early stopping with a patience of 10 and a maximum number of epochs 50 was used for the Pre-trained ResNet50. Early stopping with a patience of 20 and a maximum number of epochs 100 was used for MobileNetv2.

The ResNet34 and ResNet50 architectures were also trained using randomly initialized weights. ResNet34 was obtained. The mean pixel in the training dataset was used to center the training, validation, and test datasets. The input layer was modified to accept only one input channel in the OCT images and the output layer was changed for the classification of the three tissue types. For ResNet50, the optimizer SGD with Nesterov momentum with learning rate 0.01, momentum 0.9, and decay 0.01 was used. ResNet50 was trained with a maximum of 50 epochs, early stopping with a patience of 10, and a batch size of 32. For ResNet34, the Adam optimizer was used with learning rate 0.001, beta1 0.9, beta2 0.9999 and epsilon 1E-7. ResNet34 was trained with a maximum of 200 epochs, early stopping with a patience of 10, and a batch size of 512.

### D. Validation and Testing

A nested cross-validation and testing procedure was used to estimate the validation performance and the test performance of the models across the 10 kidneys with uncertainty quantification. The pseudo-code of the nested cross-validation and testing is shown below.

```
# 10-fold cross-testing loop
for kidney i in the 10 kidneys do
  Hold out kidney i in the test set
  # model optimization loop
  for each model configuration do
    # 9-fold cross-validation loop
  for kidney j in the remaining 9 kidneys do
    Use kidney j as the validation set
    Train a model using the remaining 8 kidneys as the training set
    Benchmark the validation performance using kidney j
    end for
    Estimate the mean validation accuracy and its standard error
  end for
  Select the best model configuration based on the validation performance
  Train a model with the selected configuration using the 9 kidneys
  Benchmark the test performance of this model using kidney i
end for
Summarize the test performance of this procedure
```

In the 10-fold cross-testing, one kidney was selected in turn as the test set. In the 9-fold cross-validation, the remaining nine kidneys were partitioned 8:1 between the training set and the validation set. Each kidney had a total of 3,000 images, including 1,000 images for each tissue type. The validation performance of a model was tracked based on its classification accuracy on the validation kidney. The classification accuracy is the percentage of correctly labeled images out of all 3,000 images of a kidney.

The 9-fold cross-validation loop was used to compare the performance of ResNet34, ResNet50, and MobileNetv2, and optimize the key hyperparameters of these models, such as pre-trained versus randomly initialized weights, learning rates, and number of epochs. The model configuration with the highest average validation accuracy was selected for the cross-testing loop. The cross-testing loop enabled iterative benchmarking of the selected model across all 10 kidneys, giving a better estimation of generalization error with uncertainty quantification.

GRAD-CAM was used to explain the predictions of a selected CNN model by highlighting the important regions in the image for the prediction outcome.

### E. OCT Imaging of Different Renal Tissues

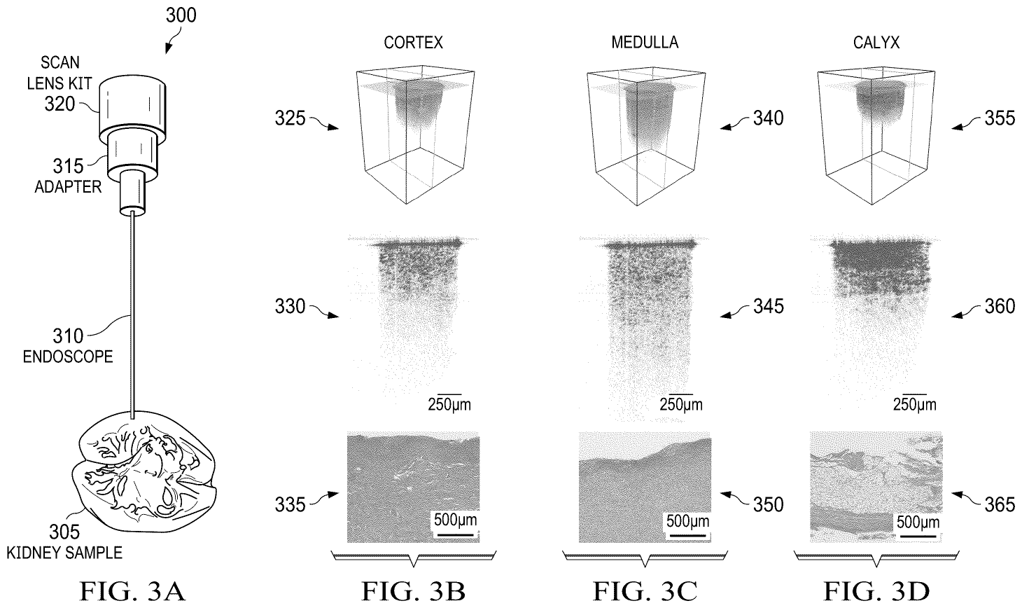

FIG. 3A is a diagram 300 of an endoscope 310 scanning a kidney sample 305. An adapter 315 stabilizes the endoscope 310 in front of an OCT scan lens kit 320. The kidney sample 305 shows different tissue types. Renal cortex is the brown tissue on the edge of the kidney sample 305. Medulla can be recognized by its red renal pyramid structures distributed on the inner side of the cortex. Calyx is featured by its obvious white structure in the central portion of the kidney sample 305. Three tissue types were imaged respectively following the procedure described above.

FIG. 3B shows 3D OCT images 325, 2D cross-sectional images 330, and histology results 335 of a renal cortex. FIG. 3C shows 3D OCT images 340, 2D cross-sectional images 345, and histology results 350 of a renal medulla. FIG. 3D shows 3D OCT images 355, 2D cross-sectional images 360, and histology results 365 of a renal calyx. FIGS. 3B-3D are featured with different imaging depth and brightness.

The renal calyx in FIG. 3B has the shallowest imaging depth, but the tissue close to the surface shows the highest brightness and density. The renal cortex in FIG. 3C and the renal medulla in FIG. 3D both present relatively homogeneous tissue structures in the 3D OCT images 340 and 355, and the imaging depth of the renal medulla in FIG. 3C is larger than the renal cortex in FIG. 3D. Furthermore, compared to the renal cortex in FIG. 3B and the renal medulla in FIG. 3D, the renal calyx in FIG. 3D was featured with horizontal stripes and a layered structure. The transitional epithelium and fibrous tissue in the renal calyx in FIG. 3D may explain the strip-like structures and significantly higher brightness in comparison to the other two renal tissues. This is the significant part for PCN insertion because the goal of PCN is to reach the calyx precisely. These imaging results demonstrated the feasibility of distinguishing the renal cortex, medulla, and calyx with the endoscopic OCT system.

### F. CNN Development and Benchmarking Results

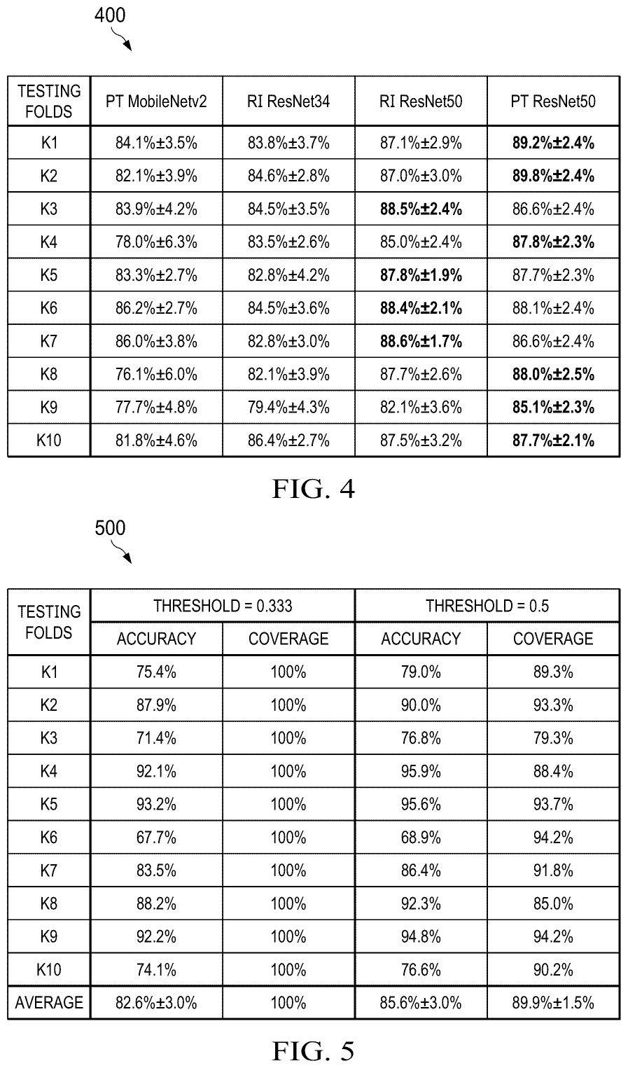

FIG. 4 is a table 400 showing the average validation accuracies and their standard errors for the PT or RI model architectures after hyperparameter optimization. RI MobileNetv2 frequently failed to learn, so only the PT MobileNetv2 model was used. The PT ResNet50 models outperformed the RI ResNet50 models in 6 of the 10 testing folds, which indicated only a small boost by the pre-training on ImageNet. For all the 10 testing folds, the validation accuracies of the ResNet50 models were significantly higher than those of the MobileNetv2 and ResNet34 models. Thus, the characteristic patterns of the three kidney tissues may require a deep CNN architecture to be recognized.

FIG. 5 is a table 500 showing the test accuracy of the best-performing model in each of the 10 testing folds. The output layer of the CNN models estimated three softmax scores that summed up to 1.0 for the three tissue types. When the category with the highest softmax score was selected for an image (i.e., a softmax score threshold of 0.333 to make a prediction), the CNN model made a prediction for every image (100% coverage) at a mean test accuracy of 82.6%. This was substantially lower than the mean validation accuracy of 87.3%, which suggested the overfitting to the validation set by the hyperparameter tuning and early stopping. The classification accuracy can be increased at the expense of lower coverage by increasing the softmax score threshold, which allowed the CNN model to make only confident classifications. When the softmax score threshold was raised to 0.5, 89.9% of the images on average were classified to a tissue type and the mean classification accuracy increased to 85.6%+3.0%. For the uncovered images, the doctors can make a prediction with the help of other imaging modality and their clinical experience.

There was substantial variability in the test accuracy among different kidneys. While three kidneys had test accuracies higher than 92% (softmax score threshold of 0.333), the kidney in the sixth fold had the lowest test accuracy of 67.7%. Therefore, the current challenge in the image classification mainly comes from the anatomic differences among the samples.

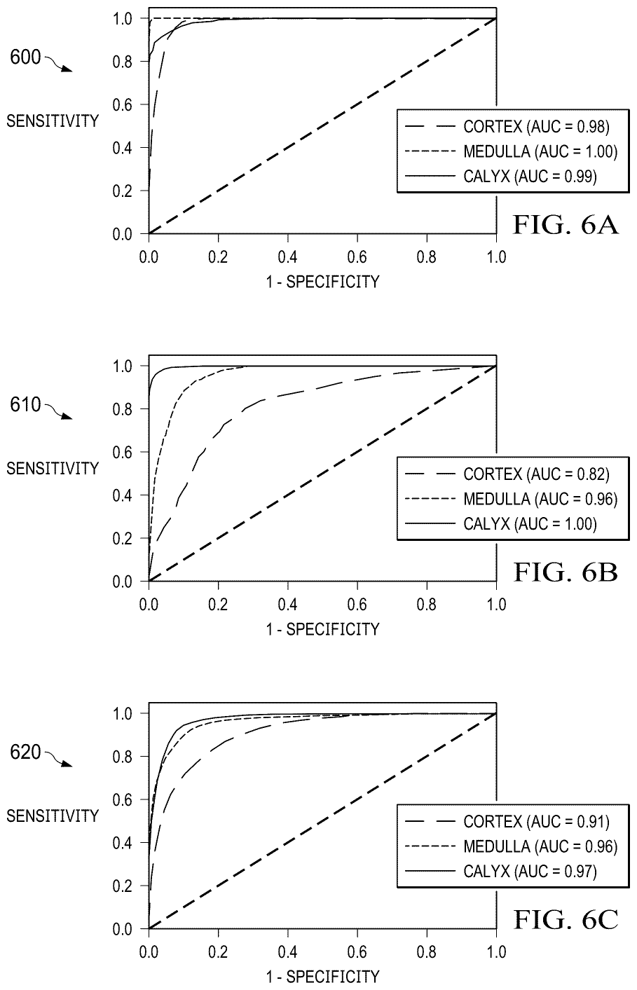

FIG. 6A is a graph 600 showing the ROC curve of the prediction results from kidney number 5. FIG. 6B is a graph 610 showing the ROC curve of the prediction results from kidney number 10. It is clear that the prediction of kidney number 5 is much more accurate than that of kidney number 10. The nested cross-validation and testing procedure was designed to simulate the real clinical setting in which the CNN models trained on one set of kidneys need to perform well on a new kidney unseen by the CNN models until the operation. When a CNN model was trained on a subset of images from all kidneys and validated on a separate subset of images from all kidneys in cross-validation as opposed to partitioning by kidneys, it achieved accuracies over 99%. This suggested that the challenge of image classification mainly stemmed from the biological differences between different kidneys. The generalization of the CNN models across kidneys can be improved by expanding the dataset with kidneys at different ages or physical conditions to represent different structural and morphological features.

FIG. 6C is a graph 620 showing average ROC curves for the three tissue types. The AUC was 0.91 for the cortex, 0.96 for the medulla, and 0.97 for the calyx.

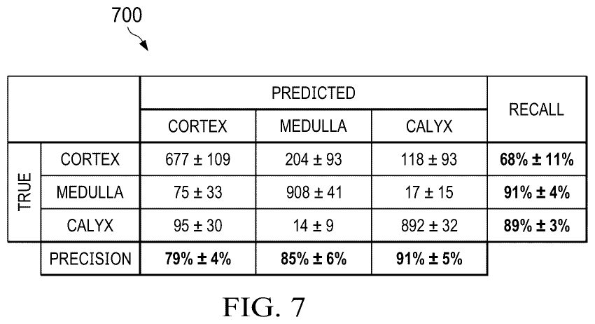

FIG. 7 is a table 700 showing the average confusion matrix for the 10 kidneys in the 10-fold cross-testing with a score threshold of 0.333 and the average recall and precision for each type of tissue. The cortex was the most challenging tissue type to be classified correctly and was sometimes mixed up with the medulla. From the original images, it was found that the penetration depths of the medulla were much larger than the cortex in seven of the ten imaged kidneys. These differences were insignificant in three other samples. This may explain the challenging classification between the cortex and the medulla.

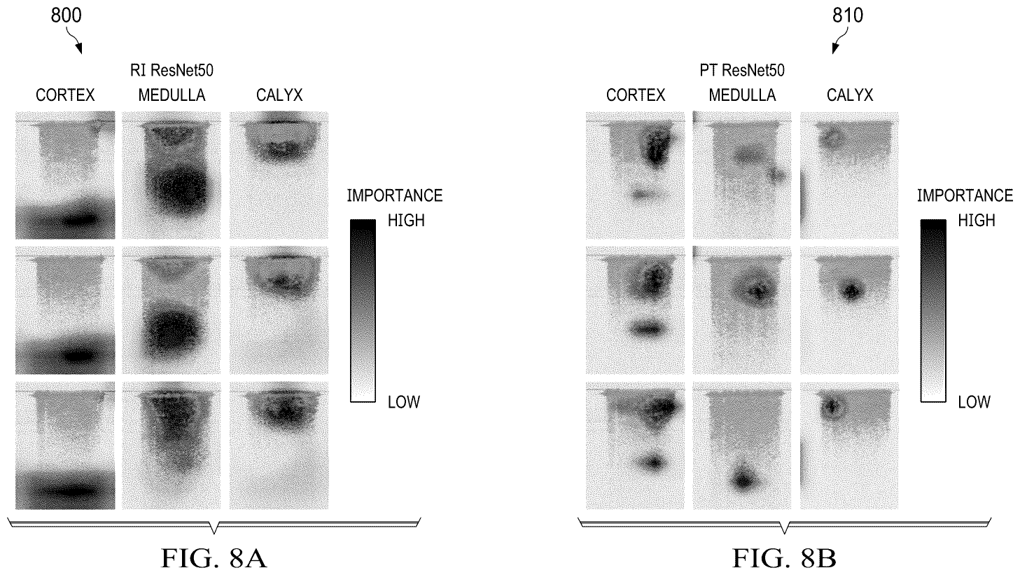

FIG. 8A shows class activation heatmaps 800 for RI ResNet50 for three representative images in each tissue type. FIG. 8B shows class activation heatmaps 810 for PT ResNet50 for three representative images in each tissue type. The class activation heatmaps 800 and 810 help demonstrate how a CNN model classified different renal types. The models and the representative images were selected from the fifth testing fold. The RI ResNet50 model performed the classification by focusing on the lower part of the images of the cortex, to both the lower part and near the upper part of the medulla images, and to the area of the calyx images with high intensity near the needle tip. The PT ResNet50 model focused on both the upper part and the lower part of the cortex images, on the middle part or the lower part of the medulla images, and on the region close to the needle tip of the calyx images. Compared to the RI ResNet50 model, the PT ResNet50 model focused more on the signal regions. The class activation heatmaps 800 and 810 provided an intuitive explanation of the classification basis for the two CNN models.

### G. Detecting Blood Vessels

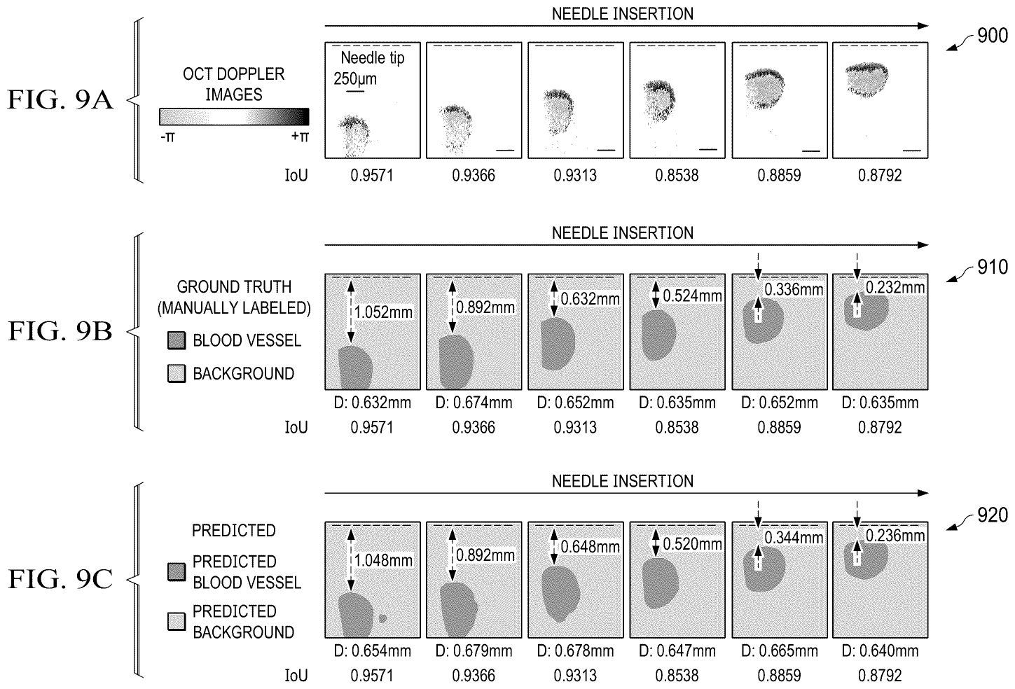

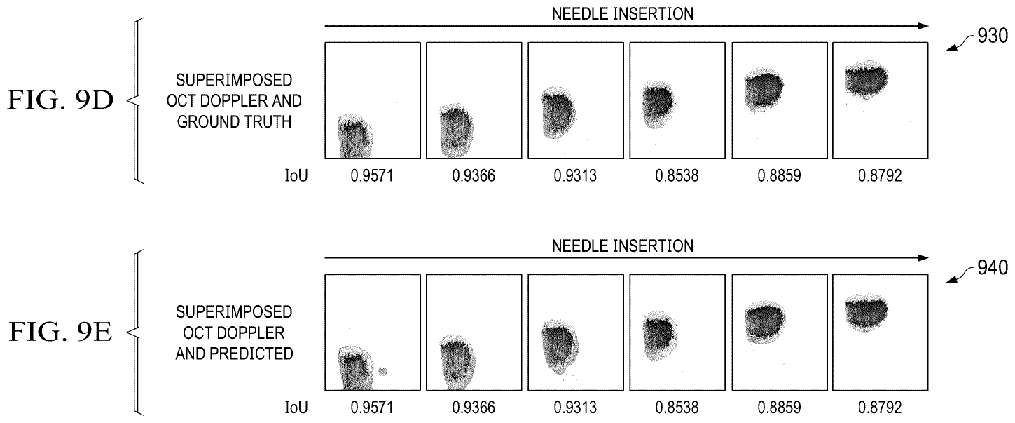

FIGS. 9A-9E show segmentation of blood vessels from Doppler OCT images. FIG. 9A shows images 900 of a blood vessel in front of a needle tip as detected by Doppler OCT. FIG. 9B shows images 910 of manually-labeled regions of blood vessels serving as a ground truth. The dashed line indicates the location of the needle tip, and “D” is the diameter of the blood vessel. FIG. 9C shows images 920 demonstrating the predicted vessel regions by nnU-net. FIG. 9D shows images 930 of superimposed OCT Doppler images with manually-labeled blood vessel regions. FIG. 9E shows images 940 of superimposed OCT Doppler images with predicted blood vessel regions by nnU-net.

The real-time blood vessel detection of the forward imaging OCT/DOCT needle in another 5 perfused human kidneys was demonstrated. During the insertion of the OCT needle into the kidney in the PCN procedure, the blood vessels in front of the needle tip were detected by Doppler OCT. FIG. 9A demonstrates a surgical scenario in which the probe encountered and compressed a ˜0.65 mm-diameter (lumen) blood vessel. The vessel was first detected at ˜1 mm distance, the upper boundary of the vessel to the needle tip, in front of the probe. The vessel was tracked as the needle advanced, and the probe was stopped prior to penetrating the vessel. Further advancing the needle compressed the vessel as shown in the far-right image in the images 900. The results demonstrated the feasibility of using DOCT imaging to detect the at-risk blood vessels during the PCN procedure using the hand-held OCT probe, without any impact of motion artifacts.

To improve the accuracy of image segmentation, a novel nnU-net framework was trained and tested using 100 2D Doppler OCT images. The blood vessels in these 100 images were first manually labeled to mark the blood vessel regions as shown in FIGS. 9B and 9D. The training and validation sets with 60 images were used to develop the nnU-net model. The segmentation accuracy of the nnU-net model was benchmarked using 40 test images. The mIoU of the predicted blood vessel pixels reached 88.46% as shown in FIGS. 9C and 9E. These new preliminary results demonstrate that with the deep-learning model, a greater than 88% mIoU can be achieved for 2D Doppler OCT images.

After obtaining the predicted regions by nnU-net as shown in FIG. 9C, an automatic image processing algorithm was developed to measure the size of the blood vessel and the distance from the needle tip. The region/area of the blood vessel was quantified by counting the pixels in the predicted regions, and the diameter was fitted by using the circular area formula because the irregular shape of the blood vessel is due to tissue compression. The distance was measured from the preset needle tip to the predicted most upper surface of the blood vessel. Compared to the ground truth shown in FIG. 9B, an accuracy (1−|Predicted −Ground Truth\/Ground Truth) of 98.7%+0.67% was achieved for distance prediction and an accuracy of 97.85%+1.34% for blood vessel diameter estimation by this algorithm.

These preliminary data clearly demonstrated at least three favorable outcomes. First, the thin-diameter forward imaging OCT/DOCT needle can detect the blood vessels in front of the needle tip in real time in the human kidney. Second, the newly developed nnU-net model can achieve >88% mloU for 2D Doppler OCT images. Third, the size and location of blood vessel can be accurately predicted. Thus, this showed a viable approach to preventing accidental blood vessel ruptures.

### H. Conclusion

The feasibility of an OCT endoscopic system for PCN surgery guidance was investigated. Three porcine kidney tissue types, the cortex, medulla and calyx, were imaged. These three kidney tissues show different structural features, which can be further used for tissue type recognition. To increase the image recognition efficiency and reduce the learning burden of the clinical doctors, CNN methods were developed and evaluated for image classification and recognition. ResNet50 had the best performance compared to ResNet34 and PT MobileNetv2 and achieved an average classification accuracy of 82.6%+3.0%.

The porcine kidneys samples were obtained from a local slaughterhouse without controlling the sample preservation and time after death. Biological changes may have occurred in the ex-vivo kidneys, including collapse of some structures of nephrons such as the renal tubules. This may have made the tissue recognition more difficult, especially the classification between the cortex and the medulla. Characteristic renal structures in the cortex can be clearly imaged by OCT in both well-preserved ex-vivo human kidneys and living kidneys and verified in an ongoing study in a lab using well-preserved human kidneys. Additionally, nephron structures distributed in the renal cortex and the medulla are different. These additional features in the renal cortex and the medulla will improve the recognition of these two tissue types and increase the classification accuracy of future CNN models when imaging in-vivo samples or well-preserved ex-vivo samples. The study established the feasibility of automatic tissue recognition using CNN and provided information for the model selection and hyper-parameter optimization in future CNN model development using in-vivo pig kidneys and well-preserved ex-vivo human kidneys.

For translating the proposed OCT probe into clinics, the endoscope will be assembled with appropriate diameter and length into the clinically-used PCN needle. In current PCN punctures, a trocar needle is inserted into the kidney. Since the trocar has a hollow structure, the endoscope can be fixed within the trocar needle. Then the OCT endoscope can be inserted into the kidney together with the trocar needle. After the trocar needle tip arrives at the destination (such as the kidney pelvis), we will withdraw the OCT endoscope from the trocar needle and other surgical processes can be continued. During the whole puncture, no extra invasiveness will be caused. Since the needle will keep moving during the puncture, there will be a tight contact between the needle tip and the tissue. Therefore, the blood, if any, will not accumulate in front of the needle tip. From previous experience in the in-vivo pig experiment guiding the epidural anesthesia using the OCT endoscope, the presence of blood is not a substantial issue. The diameter of the GRIN rod lens used in the study was 1.3 mm. In the future, the current setup will be improved with a smaller GRIN rod lens that can be fit inside the 18-gauge PCN needle, which is clinically used in the PCN puncture. Furthermore, the GSM device will be miniaturized based on MEMS technology, which will enable ease of operation and is important for translating the OCT endoscope to clinical applications. The current employed OCT system has a scanning speed up to 200 kHz, and the 2D tissue images in front of the PCN needle can be provided to surgeons in real time. Using ultra-high-speed laser scanning and a data processing system, 3D images of the detected sample can be obtained in real time. In the next step, 3D images that further improve classification accuracy may be acquired because of the added information content in 3D images.

### I. Exemplary Method

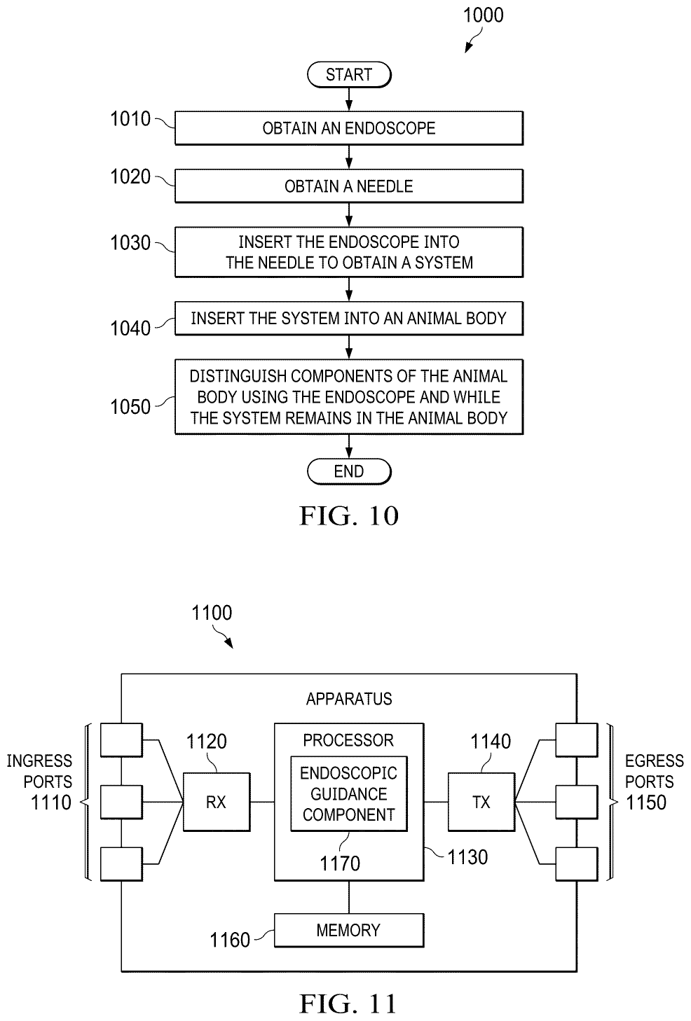

FIG. 10 is a flowchart illustrating a method 1000 for endoscopic guidance using neural networks according to one non-limiting embodiment of the present disclosure. At step 1010, an endoscope is obtained. At step 1020, a needle is obtained. At step 1030, the endoscope is inserted into the needle to obtain a system. At step 1040, the system is inserted into an animal body. Finally, at step 1050, components of the animal body are distinguished using the endoscope and while the system remains in the animal body.

The method 1000 may comprise additional embodiments. For instance, the method 1000 further comprises training a CNN to distinguish the components. The method 1000 further comprises further training the CNN to distinguish among a cortex of a kidney, a medulla of the kidney, and a calyx of the kidney. The method 1000 further comprises further training the CNN to distinguish blood vessels from other components. The method 1000 further comprises incorporating the CNN into the endoscope. The CNN comprises an input layer, a convolutional layer, a max-pooling layer, a flatten layer, dense layers, and an output layer. The animal body is a human body. The method 1000 further comprises further distinguishing a calyx of a kidney from a cortex of the kidney and a medulla of the kidney; inserting, based on the distinguishing, the system into the calyx; and removing the endoscope from the system to obtain the needle. The method 1000 further comprises further distinguishing the calyx from a blood vessel; and avoiding contact between the system and the blood vessel. The method 1000 further comprises removing kidney stones while the needle remains in the calyx. The method 1000 further comprises inserting, based on the distinguishing, the system into a kidney of the animal body; and obtaining a biopsy of the kidney. The system is a forward-view endoscopic OCT system.

### J. Exemplary Computing Apparatus

FIG. 11 is a schematic diagram of an apparatus 1100 according to one non-limiting embodiment of the present disclosure. The apparatus 1100 may implement the disclosed embodiments. The apparatus 1100 comprises ingress ports 1110 and an RX 1120 to receive data; a processor 1130, or logic unit, baseband unit, or CPU, to process the data; a TX 1140 and egress ports 1150 to transmit the data; and a memory 1160 to store the data. The apparatus 1100 may also comprise OE components, EO components, or RF components coupled to the ingress ports 1110, the RX 1120, the TX 1140, and the egress ports 1150 to provide ingress or egress of optical signals, electrical signals, or RF signals.

The processor 1130 is any combination of hardware, middleware, firmware, or software. The processor 1130 comprises any combination of one or more CPU chips, cores, FPGAs, ASICs, or DSPs. The processor 1130 communicates with the ingress ports 1110, the RX 1120, the TX 1140, the egress ports 1150, and the memory 1160. The processor 1130 comprises an endoscopic guidance component 1170, which implements the disclosed embodiments. The inclusion of the endoscopic guidance component 1170 therefore provides a substantial improvement to the functionality of the apparatus 1100 and effects a transformation of the apparatus 1100 to a different state. Alternatively, the memory 1160 stores the endoscopic guidance component 1170 as instructions, and the processor 1130 executes those instructions.

The memory 1160 comprises any combination of disks, tape drives, or solid-state drives. The apparatus 1100 may use the memory 1160 as an over-flow data storage device to store programs when the apparatus 1100 selects those programs for execution and to store instructions and data that the apparatus 1100 reads during execution of those programs. The memory 1160 may be volatile or non-volatile and may be any combination of ROM, RAM, TCAM, or SRAM.

A computer program product may comprise computer-executable instructions for storage on a non-transitory medium and that, when executed by a processor, cause an apparatus to perform any of the embodiments. The non-transitory medium may be the memory 1160, the processor may be the processor 1130, and the apparatus may be the apparatus 1100.

### K. Embodiments

In an embodiment, a method comprises: obtaining an endoscope; obtaining a needle; inserting the endoscope into the needle to obtain a system; inserting the system into an animal body; and distinguishing components of the animal body using the endoscope and while the system remains in the animal body.

The method may comprise additional embodiments. For instance, the method further comprises training a CNN to distinguish the components. The method further comprises further training the CNN to distinguish among a cortex of a kidney, a medulla of the kidney, and a calyx of the kidney. The method further comprises further training the CNN to distinguish blood vessels from other components. The method further comprises incorporating the CNN into the endoscope. The CNN comprises an input layer, a convolutional layer, a max-pooling layer, a flatten layer, dense layers, and an output layer. The animal body is a human body. The method further comprises: further distinguishing a calyx of a kidney from a cortex of the kidney and a medulla of the kidney; inserting, based on the distinguishing, the system into the calyx; and removing the endoscope from the system to obtain the needle. The method further comprises: further distinguishing the calyx from a blood vessel; and avoiding contact between the system and the blood vessel. The method further comprises removing kidney stones while the needle remains in the calyx. The method further comprises: inserting, based on the distinguishing, the system into a kidney of the animal body; and obtaining a biopsy of the kidney. The system is a forward-view endoscopic OCT system.

A system comprises: a needle; and an endoscope inserted into the needle and configured to: store a CNN; distinguish among a cortex of a kidney of an animal body, a medulla of the kidney, and a calyx of the kidney using the CNN; and distinguish between vascular tissue and non-vascular tissue in the animal body using the CNN.

The system may comprise additional embodiments. For instance, the CNN comprises an input layer, a convolutional layer, a max-pooling layer, a flatten layer, dense layers, and an output layer. The animal body is a human body. The system is a forward-view endoscopic OCT system. The endoscope has a diameter of about 1.3 mm. The endoscope has a length of about 138.0 mm. The endoscope is configured to have a view angle of 11.0°. The needle is configured to remove a kidney stone from the kidney or obtain a biopsy of the kidney.

### II. Computer-Aided Veress Needle Guidance Using Endoscopic OCT and CNNs

During laparoscopic surgery, the Veress needle is commonly used in pneumoperitoneum establishment. Precise placement of the Veress needle is still a challenge for the surgeon. In this study, a computer-aided endoscopic OCT system was developed to effectively and safely guide Veress needle insertion. This endoscopic system was tested by imaging subcutaneous fat, muscle, abdominal space, and the small intestine from swine samples to simulate the surgical process, including the situation with small intestine injury. Each tissue layer was visualized in OCT images with unique features and subsequently used to develop a system for automatic localization of the Veress needle tip by identifying tissue layers or spaces and estimating the needle-to-tissue distance. CNNs were used in automatic tissue classification and distance estimation. The average testing accuracy in tissue classification was 98.53+0.39%, and the average testing relative error in distance estimation reached 4.42+0.56% (36.09+4.92 μm).

### A. Introduction

Laparoscopy is a modern and minimally invasive surgical technique used for diagnostic and therapeutic purposes. With the development of video cameras and other medical auxiliary instruments, laparoscopy has become a procedure widely used in various surgeries such as cholecystectomy, appendectomy, herniotomy, gastric banding, and colon resection. In the first step of the laparoscopic procedure, a trocar/Veress needle is inserted into the patient's abdominal cavity through a small skin incision. The Veress needle penetrates subcutaneous fat and muscle before reaching the abdominal cavity. Once entry to the peritoneal cavity has been achieved, gas insufflation is used to establish pneumoperitoneum for the next surgical process. The pneumoperitoneum establishment step does not take a long time; however, more than 50% of all laparoscopic procedure complications occur during this step. In most of the current practices, the Veress needle is blindly inserted, and appropriate needle positioning is largely dependent on prior experience from the surgeon. Complications such as subcutaneous emphysema, gas embolism, or injury to internal organs during abdominal entry could happen when the Veress needle is not appropriately inserted. While the average incidence rate of severe needle injury is below 0.05%, there are more than 13 million laparoscopic procedures performed annually worldwide. Thousands of patients suffer from needle insertion injuries each year. The most common injuries are lesions of abdominal organs, especially small intestines.

Imaging methods have been proposed in guiding the Veress needle insertion. For instance, ultrasound has been used to visualize the different layers of abdominal wall in guiding the Veress needle insertion. MRIs have been utilized to accurately measure the Veress needle insertion depth. In addition, virtual reality techniques have been proved to be a useful tool for Veress needle insertion. Nevertheless, these techniques cannot accurately locate the needle tip because of the limited resolution and tissue deformation during needle insertion. Therefore, new techniques that can better guide the Veress needle are critically needed.

(OCT is an established biomedical imaging technique that can visualize subsurface tissue. OCT provides high axial resolution at ˜10 μm and several millimeters' imaging depth, thus OCT has the potential for providing better imaging quality in Veress needle guidance. However, benchtop OCT cannot be directly used for Veress needle guidance due to the limited penetration depth. Endoscopic OCT systems have been applied in many surgical guidance procedures such as the investigation of colon cancer, vitreoretinal surgery, and nasal tissue detection. In one approach, an OCT endoscope based on GRIN rod lens demonstrated its feasibility in real-time PCN guidance and epidural anesthesia guidance.

Below, the OCT endoscope is adapted for Veress needle guidance. To simulate the laparoscopy procedure, the endoscopic OCT system was used to image different tissue layers including subcutaneous fat, muscle, abdominal space, and small intestine from swine abdominal tissue. These tissues can be recognized based on their distinct OCT imaging features. To assist doctors, CNNs were developed for recognizing the different types of tissues and estimating the exact distance between needle tip and the small intestine from OCT images. OCT images were taken from four tissue layers (subcutaneous fat, muscle, abdominal space, and small intestine) along the path of the Veress needle. These images were then used to train and test a classification model for tissue layer recognition and a regression model for estimation of the distance from the tip of the needle to the small intestine. The CNN architectures used for the classification and regression tasks included ResNet50, InceptionV3, and Xception. Results from these three architectures were analyzed and benchmarked. Thus, an endoscopic OCT system is combined with CNN as an imaging platform for guiding the Veress needle procedure.

### B. Experimental Setup

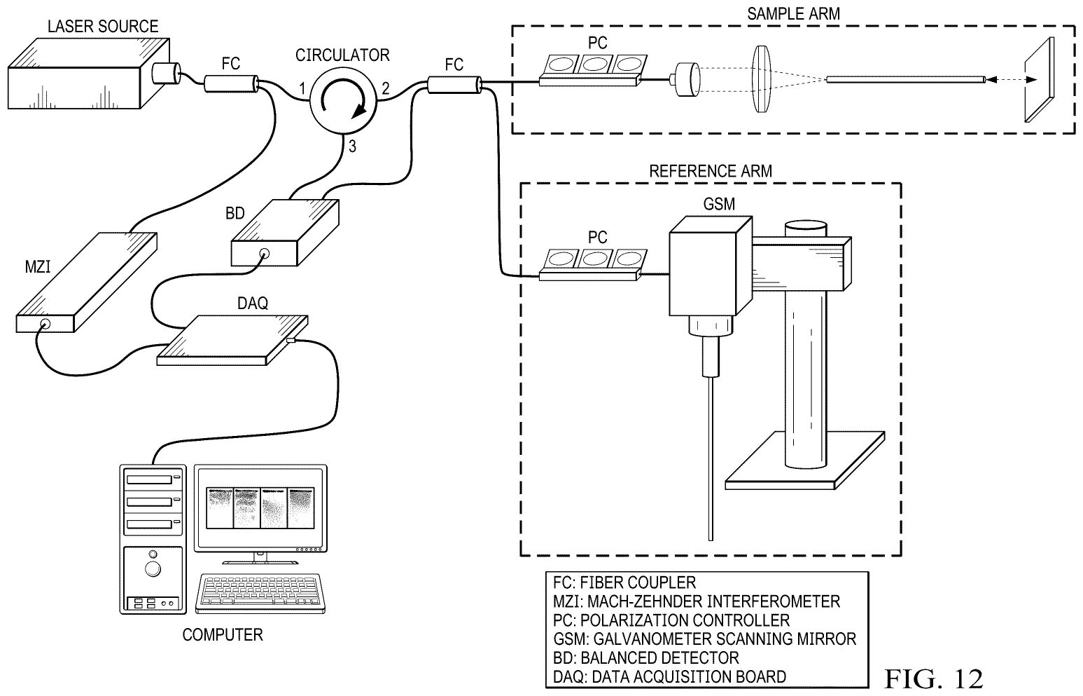

FIG. 12 is a schematic of an endoscopic OCT system for Veress needle guidance. The endoscopic OCT system was established on an SS-OCT system, which applied a laser source with a 1,300 nm center wavelength and 100 nm bandwidth. The laser source provided output power of around 25 mW. The system had the axial scanning rate of up to 200 kHz. The light was initially split by a coupler into two different parts, one of which accounted for 97% of the total power and the other one took the remaining 3%. An MZI received the 3% power and generated a frequency-clock signal to trigger the imaging sampling process. The 97% light was further transmitted to an optical circulator. When the light exited from port 2, it was split evenly into the sample arm and reference arm of the OCT system. Polarization controllers were assembled in each coherence arm to control the noise levels. The interference signal of the backscattered light from sample arm and the reflected beam from reference arm was sent to the BD for further noise reduction, and then to the DAQ board and computer for post processing. Cross-sectional OCT images from different imaging depths of the sample can be provided through Fourier transform.

To build the endoscopic system, GRIN rod lens were used as endoscopes. One GRIN lens was stabilized in front of the galvanometer scanner as demonstrated in FIG. 12. The GRIN lens transmitted the imaging information from the distal end to the proximal end while the spatial resolution remained constant. The proximal surface of the GRIN lens was placed at the focal point of the OCT scanner to acquire the tissue images in front of the GRIN lens. To compensate for light dispersion, another identical GRIN lens was placed into the light path of the reference arm. The GRIN lenses had a length of 138 mm and a diameter ˜1.30 mm. Stainless steel tubes were assembled to protect the GRIN lenses. Lateral FOV of the system reached ˜1.25 mm, and the sensitivity was calibrated to be ˜92 dB. The endoscopic OCT achieved ˜11 μm axial resolution and 20 μm transverse resolution.

### C. Data Acquisition

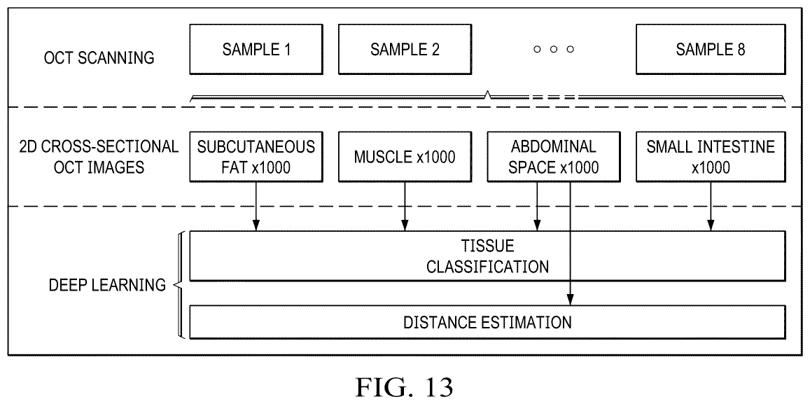

The OCT images of subcutaneous fat, muscle, abdominal space, and small intestine were taken from eight pigs. For each sample, 1,000 2D OCT cross-sections were selected. A total of 32,000 images were utilized for CNN tissue classification model development. For the distance estimation task, there were a total of 8,000 OCT images of abdominal space used, and distance between the GRIN lens end and the small intestine varied in these images. FIG. 13 is a flow diagram of data acquisition and processing.

The original size of each image was 320 (transverse/X axis)×480 (longitudinal axis/depth/Z direction) pixels. The pixel size on both axes were 6.25 μm. To decrease the computation burden, the size of the 2D images was reduced by cropping the unnecessary part of the images' edges. They were cropped to 216×316 pixels for tissue classification and 180×401 pixels for distance estimation.

### D. CNN Method for Tissue Classification

CNNs were used to accomplish the task of identifying the layer of tissues in which an image was taken. The four layers analyzed for classification include fat, muscle, abdominal space, and intestine. For each subject, there were 1,000 images taken from each tissue layer, and the layer in which the image was taken was manually annotated and represented the ground truth label for the classification task. The CNN model architectures used for model development included ResNet50, InceptionV3 and Xception, which contained 25.6 million, 23.9 million, and 22.9 million parameters, respectively. Training took place over 20 epochs with a batch size of 32. A cross-entropy optimizer was used with Nesterov momentum, a learning rate of 0.9, and a decay rate of 0.01. The loss function was sparse categorical cross-entropy, and accuracy was used as the primary evaluation metric for the tissue classification task. ResNet50, Xception, and Inception V3 were used because of their demonstrated performance on similar image prediction tasks and relatively comparable network depths. Initially, a wide range of architectures were selected and tested, including EfficientNet (B3, B4, and B5), InceptionV3, NasNetLarge, NasNetMobile, ResNet50, ResNet101, and Xception. On average, the ResNet50, Xception, and InceptionV3 architectures supported the best performing models in regard to accuracy and efficiency. The accuracy was calculated as follows:

$$Accuracy = \frac{T P + T N}{T P + T N + F P + F N} . \qquad ( 1 )$$

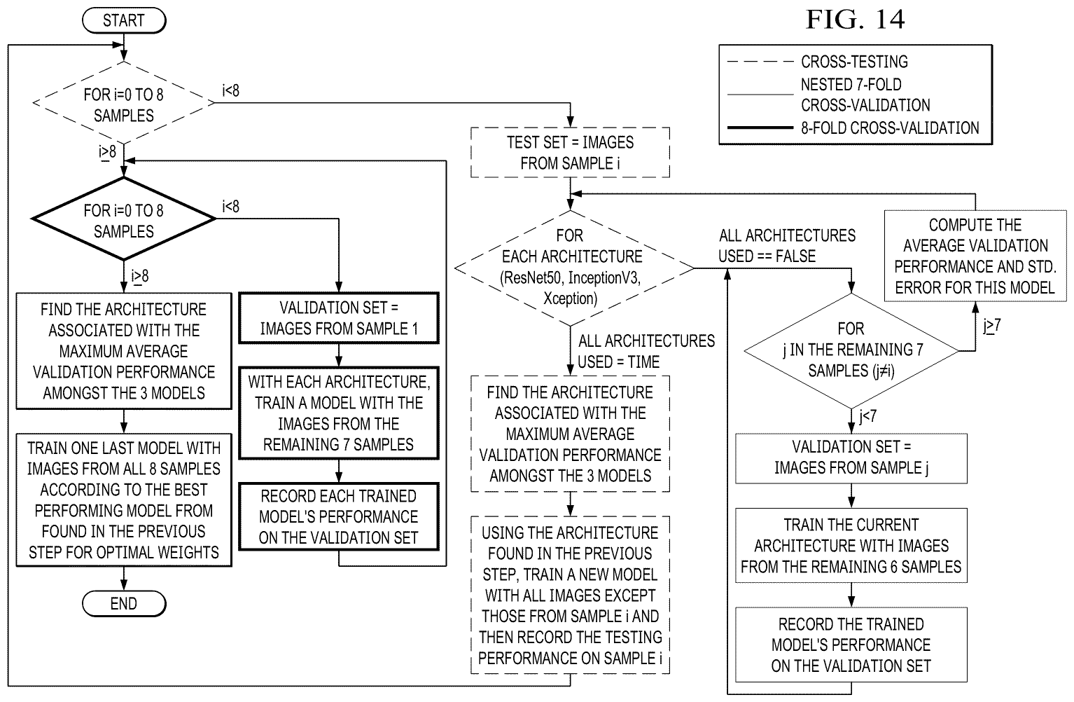

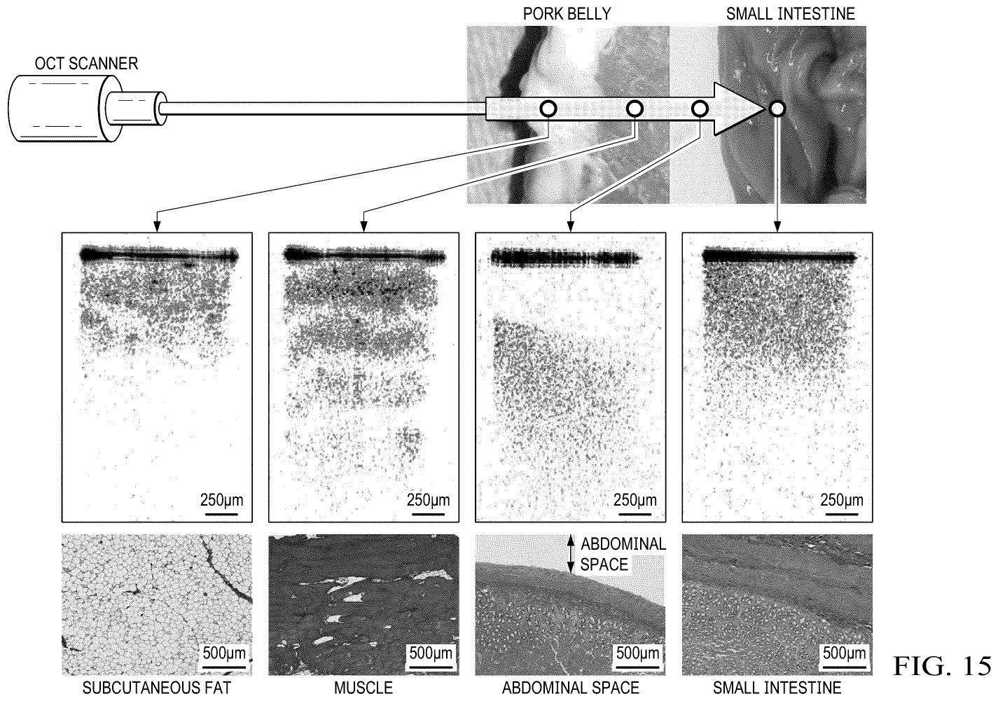

FIG. 14 is a flowchart of a process of nested cross-validation, cross-testing, and 8-fold cross-validation. Specifically, nested cross-validation and cross-testing were used to select an optimal model and evaluate the performance. For nested cross-validation and testing, images were separated into eight folds based on the subject in which the images were taken. With nested cross-validation, the performance of the three model architectures were compared, and the architecture with the highest accuracy was selected for the corresponding testing phase. Once the optimal model architecture was determined for a given nested cross-validation fold, the cross-testing phase required training a new model with all images except those from the corresponding testing fold, and the new model's accuracy on the unseen testing images was recorded. Training and testing took place on ten compute nodes containing GPUs on the Summit supercomputer at Oak Ridge National Laboratory.

### E. CNN Regression for Distance Measurement

Regression CNNs were used to estimate the distance from the Veress needle lens to the intestine. The distance from the needle tip to the intestine represented the ground truth label and was manually annotated.

CNN regression models were constructed with the same three architectures used for classification, which includes ResNet50, InceptionV3, and Xception, and the same nested cross-validation and cross-testing approach was used for training and performance evaluation. For the regression model architecture, the final output layer was changed to a single neuron with an identity activation function. Training involved using the SGD optimization algorithm, a learning rate of 0.01, decay rate of 0.09, and Nesterov momentum. Training took place over 20 epochs with a batch size of 32. The loss function was the MAPE. Regression model development was accomplished on a private workstation containing two NVIDIA RTX 3090 GPUs. Here, MAPE and MAE were utilized to evaluate the estimation accuracy of the distance and were calculated as follows:

$$MAPE ( \% ) = \frac{100 \%}{n} {\sum }_{i = 1}^{n} \frac{\vert {Y}_{i} - {X}_{i} \vert}{{Y}_{i}} \qquad ( 2 )$$

$$MAE = \frac{1}{n} {\sum }_{i = 1}^{n} \vert {Y}_{i} - {X}_{i} \vert , \qquad ( 3 )$$

Where Xᵢ was the estimated value, Yᵢ was the manually-labelled ground truth value, and n was the number of the images.

### F. Imaging Results of Endoscopic OCT System

The subcutaneous fat, muscle, abdominal space, and small intestine tissues from eight different swine samples were used to mimic the practical tissue layers that the Veress needle traverses. Fat and muscle were both taken from the abdominal areas. In the experiment, the GRIN lens in the sample arm was inserted into the three different tissues for imaging. Moreover, to replicate the condition when the needle tip was in the abdominal cavity, the needle tip was kept at different distances in front of the small intestine and took the OCT images as the abdominal space layer.

FIG. 15 shows examples of 2D OCT results of the three different tissues and abdominal space. The 2D results demonstrated clear differences between each other: Abdominal space could be easily recognized from the gap between the tip of the GRIN lens and the small intestine tissue on the bottom. Among the three tissues, muscle had clear transverse fiber structures which was shown as different light and dark layers on the OCT image and had the largest penetration depth. The imaging result of the small intestine showed homogeneous tissue density and brightness. Regarding the subcutaneous fat images, some granular structures occurred because of the existence of adipocytes. The corresponding histology results were also included. Different tissues presented distinct cellular structures and distributions and correlated well with their OCT images. These results proved the potential of using OCT to distinguish different tissue layers.

### G. Multi-Class Classification

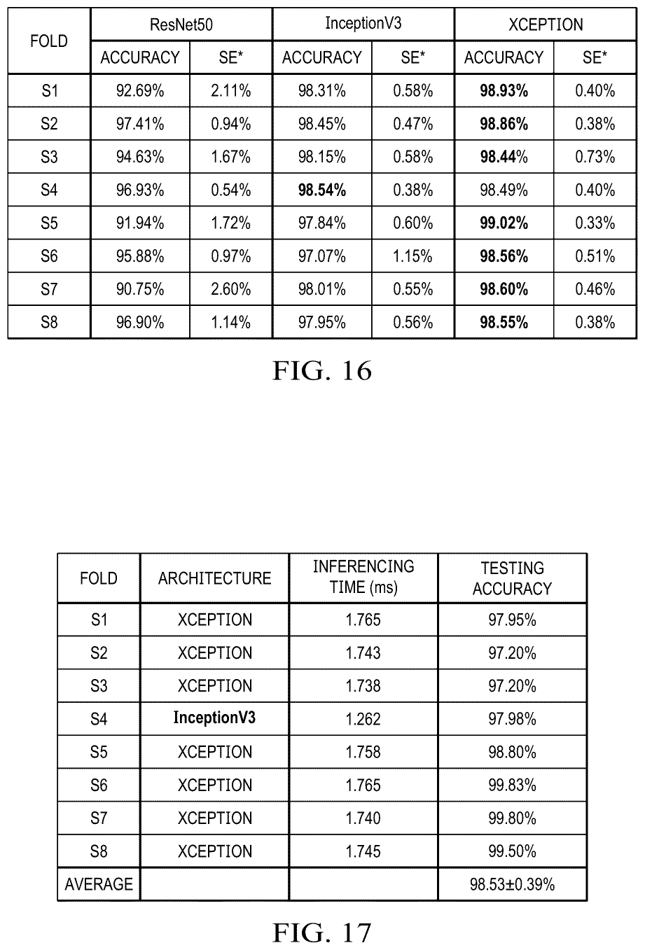

There were 32,000 total images with the size of 216×316 pixels taken from eight subjects used for training and testing the tissue classification model. Three CNN models were applied: ResNet50, Inception V3 and Xception. FIG. 16 is a tables of the average nested 7-fold cross-validation accuracies for tissue-layer classification. All models achieved 90% accuracy or higher on the validation set. For InceptionV3 and Xception models, the average accuracies were both higher than 97%.

Cross-testing was further performed to provide an unbiased evaluation of the classification performance on the set of test images (i.e., images that were not used during nested cross-validation). The architecture associated with the highest nested cross-validation accuracy in each cross-validation fold was used to train a new model for the corresponding cross-testing fold. During cross-testing, a new model was trained with images from seven subjects (7,000 images) in both the training and validation folds and tested with images from one subject (1,000 images) in the test fold. FIG. 17 is a table of the 8-fold cross-testing accuracies on the testing set in each testing fold. The testing results along with inferencing times are shown. The best testing accuracy on the testing images was 99.825% in the S6 testing fold. There was a tie for the lowest testing accuracy of 97.200% in the S2 and S3 testing fold. The classification inferencing time is the amount of time it took for the classification model to predict the tissue layer on a single image. The inferencing time for Xception was slightly greater than that of Inception V3.

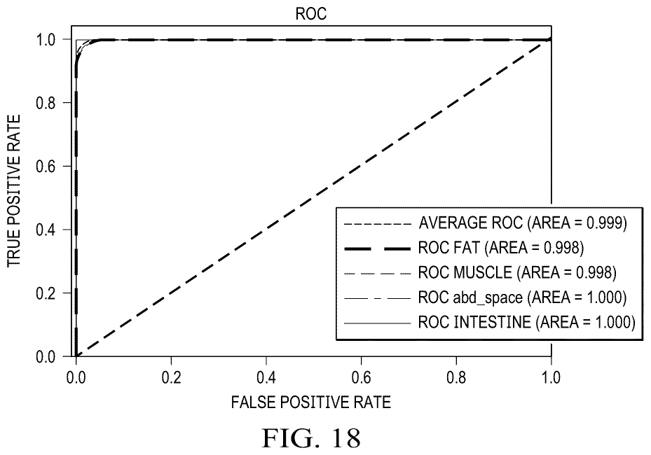

From the classification cross-testing benchmarking results, the Xception architecture was used in 7 out of the 8 cross-testing folds, and the InceptionV3 architecture was used once (selected via cross-validation). FIG. 18 is a graph of aggregated ROC across all 8 testing folds using the Xception model. The eight testing folds include 32,000 images. The ROC curve shows that the models were able to classify the images pertaining to each tissue layer with high accuracy. The average ROC AUC score was 0.998967 and the r2 value was 0.9839.

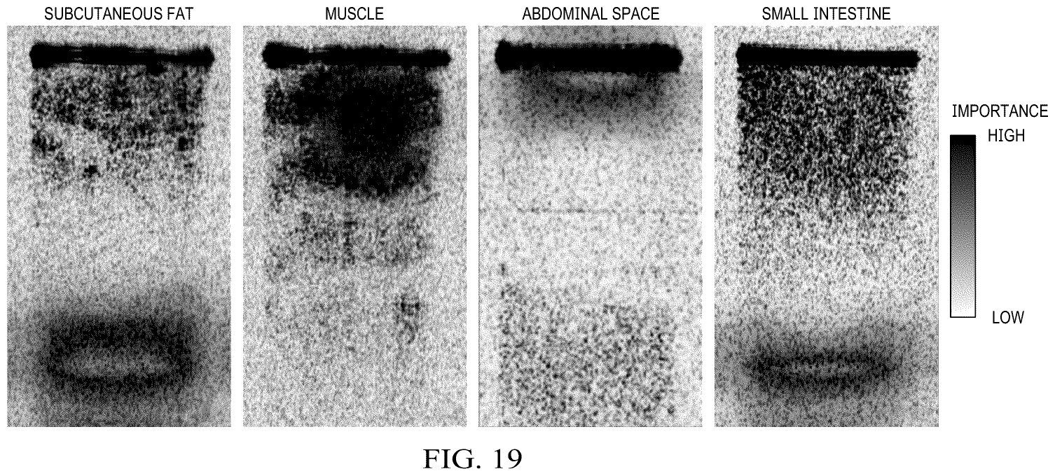

FIG. 19 are heatmaps of tissue classification activation obtained from sample 1. FIG. 19 has a scale bar of 250 μm. FIG. 19 visually explains the model predictions of the different tissue layers. The activation distributions differed in position and size among the four tissue layer types. In subcutaneous fat and small intestine, the activation was mainly concentrated in the bottom of the images. However, the size of the activated area on the image of subcutaneous fat was larger than the activated area on the image of the small intestine, and there was also increased attention given to the area right below the needle tip. For muscle and abdominal space, the activated areas on the image were near the top, but there was greater focus given to the area just under the needle tip in muscle images. In contrast to the muscle images, the activated areas of the abdominal space images were more concentrated over the needle tip.

After the performance of the model development procedure was benchmarked by the cross-testing, a final model was generated using this procedure in two steps. First, an architecture was selected using 8-fold cross-validation. As expected, the Xception architecture provided higher accuracy on average (98.37+0.59%) than ResNet50 (95.92+1.40%) and InceptionV3 (97.46+0.75%). Then, a final model was trained using all 8 folds with the Xception architecture.

### H. Distance Estimation

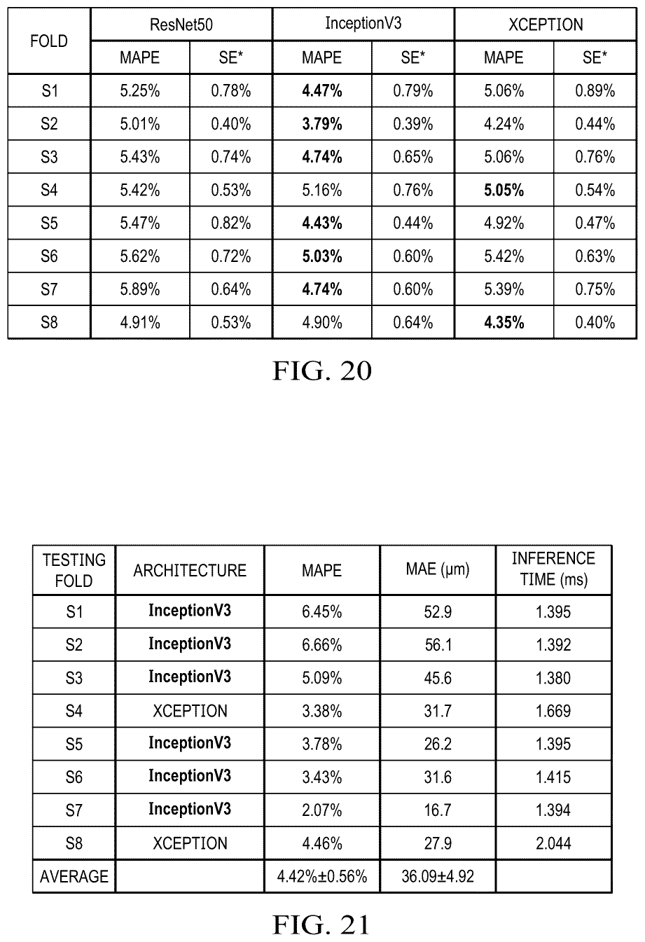

There were 8,000 images in total used for the regression task, including 1,000 images per subject. The images of the abdominal space were taken at a range of distances between approximately 0.2 mm and 1.5 mm from the needle tip to the intestine. To estimate the distance values between the needle tip and the surface of small intestine, the same three architectures (ResNet50, Inception V3, and Xception) were also utilized for the regression task. The MAPE was used to evaluate the distance estimation error. Nested cross-validation and cross-testing were performed in the same fashion as for the tissue-layer classification task. FIG. 20 is a table showing the average nested cross-validation MAPE with standard error in each nested cross-validation fold. The distance estimation nested cross-validation results indicate that the Inception V3 achieved the lowest average error in six out of eight folds, and Xception achieved the lowest average error in the other two folds.

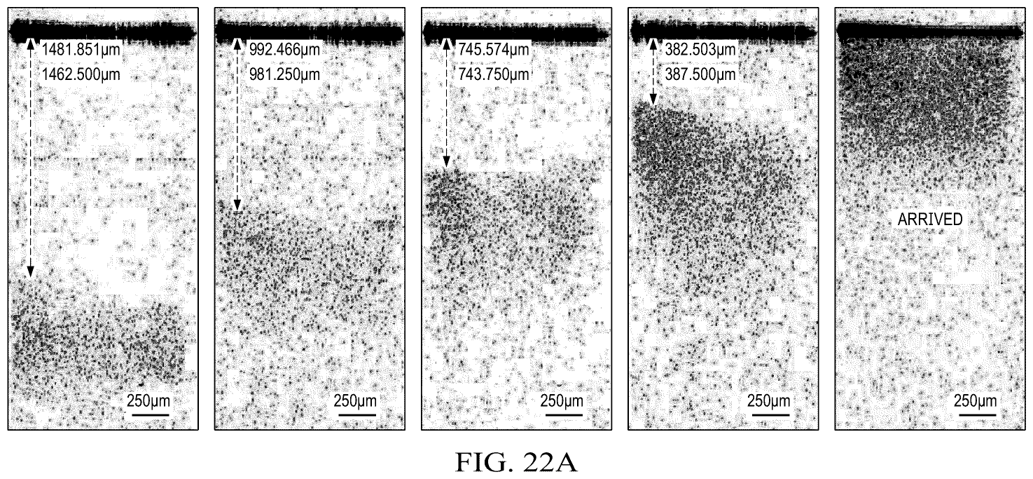

FIG. 21 is a table of MAPE with SE and MAE with SE during 8-fold cross-testing. Similar to tissue classification, cross-testing was used to get an unbiased evaluation of the performance on the distance estimation task. InceptionV3 provided the highest MAPE of 6.66% with MAE of 56.1 μm in fold S2, and the lowest MAPE of 2.07% with MAE of 16.7 μm in fold S7. Overall, the MAPEs were all under 7%, and the MAEs never exceeded 60 μm.

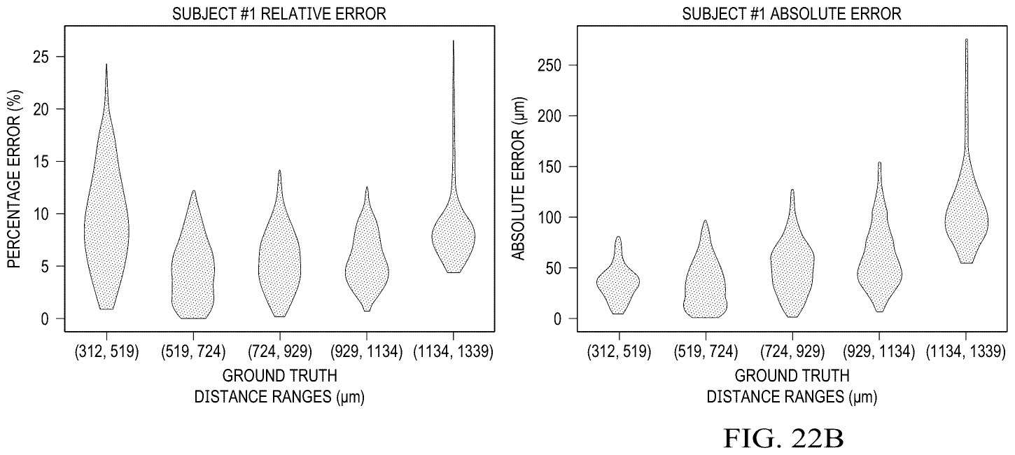

FIG. 22A shows comparisons between the manually labeled results (ground truth value) and predicted results. FIG. 22B shows violin plots from sample one. The violin plots show the error distribution based on distance.

After the regression model development procedure was benchmarked by cross-testing, this procedure was repeated to produce a final model. Because no data needed to be held back for testing, all 8 folds were used in the cross validation for final architecture selection. The models trained with the InceptionV3 architecture provided lower error on average (4.44+0.43%) than ResNet50 (5.29+0.56%) and Xception (4.77+0.58%). After the architecture was selected, because no data needed to be held back for validation or testing, all 8 folds were used to train the final model.

### I. Discussion

The feasibility of the forward-view endoscopic OCT system for Veress needle guidance is demonstrated. Compared to other imaging methods, OCT can provide more structural details of the subsurface tissues to help recognize the tissue type in front of the needle tip. Four tissue layers following the sequence of the tissues that the Veress needle passed through during the surgery were imaged by the endoscopic OCT system, including subcutaneous fat, muscle, abdominal space, and small intestine. The OCT images of these four layers could be distinguished by their unique imaging features. By fitting the rigid OCT endoscope inside the hollow bore of the Veress needle, no additional invasiveness will be introduced from the OCT endoscope. The OCT endoscope will provide the images of the tissues in front of the Veress needle during insertion, thus indicating the needle tip location in real time and facilitating the precise placement of the Veress needle.

Deep learning was used to automate the OCT imaging data processing. Three CNN architectures, including ResNet50, InceptionV3 and Xception, were cross-validated for both tasks. These three architectures were used because of their demonstrated performance on similar image prediction tasks and relatively comparable network depths. nnU-Net is another deep learning model that may be used. Initially, a wide range of architectures were selected and tested, including EfficientNet (B3, B4, and B5), Inception V3, NasNetLarge, NasNetMobile, ResNet50, ResNet101, and Xception. On average, the ResNet50, Xception, and Inception V3 architectures supported the best performing models in regard to accuracy and efficiency. Among these three architectures, the best architecture was found to be Xception for tissue layer classification and InceptionV3 for estimating the distance from needle tip to small intestine surface. However, all three architectures provided very high prediction performance and had only insignificant performance differentials among them. Nested cross-validation and cross-testing were used to provide an unbiased performance benchmarking of the model development procedure from architecture selection to model training. The average testing accuracy of the procedure was 98.53+0.39% for tissue layer classification. The average MAPE of the procedure was 4.42%+0.56% for the distance estimation.

For the classification task, the training time per fold over 28,000 images was ˜98 minutes on average for the Xception architecture and ˜32 minutes for the Inception V3 architecture during cross testing. The average inferencing time (i.e., the time it took for a trained model to make a prediction on a single image) for the Xception models during cross-testing was 1.75 ms, while the InceptionV3 model had an interpretation time of 1.26 ms. The classifiers were trained and tested using NVIDIA Volta GPUs. For the regression task, the average training time for the InceptionV3 model during cross-testing was ˜367 minutes, which took place over 7,000 images. The average training time for the Xception model was ˜1,244 minutes. The average interpretation time for the Inception V3 models was 1.30 ms. And the average interpretation time for the Xception model was ˜1.86 ms. Regression model training and testing took place on NVIDIA RTX 3090 GPUs.

The feasibility of using endoscopic OCT and deep learning methods in Veress needle guidance was shown. The system will be applied in in-vivo swine experiments. Blood flows exist during in-vivo experiments. Except for the lesions of abdominal organs, injury to the blood vessels is also a major complication in the Veress needle insertion. Major vascular injuries, especially to aorta, vena, cava or iliac vessels, are risky to patients' lives during Veress needle insertion. The mortality rate can reach up to 17% when injuries to large vessels happen. Doppler OCT is an extension of the current OCT endoscope that can help detect flows. Doppler OCT endoscope has been used for detecting the at-risk blood vessels within sheep brain in real time, in colorectal cancer diagnosis, management of pulmonary nodules, and human GI tract imaging and treatment. Therefore, the proposed OCT endoscope has the potential to solve the problem of blood vessel injury during Veress needle insertion. As to hardware, the OCT scanner can be redesigned to make it easier for surgeons to operate. The models can be improved through knowledge distillation and weight pruning. Furthermore, since the proposed OCT endoscope system can distinguish different tissue types in front of the needle tip, it also has potential for guiding other needle-based interventions such as PCN needle guidance in kidney surgery, epidural anesthesia imaging guidance in painless delivery, the tumor tissue detection in cancer diagnosis, and a variety of needle biopsy procedures.

### III. Epidural Anesthesia Needle Guidance by Forward-View Endoscopic OCT and Deep Learning

Epidural anesthesia requires injection of anesthetic into the epidural space in the spine. Accurate placement of the epidural needle is a major challenge. To address this, a forward-view endoscopic OCT system for real-time imaging of the tissue in front of the needle tip during the puncture is developed. This OCT system was tested in porcine backbones, and a set of deep learning models was developed to automatically process the imaging data for needle localization. A series of binary classification models was developed to recognize the five layers of the backbone, including fat, interspinous ligament, ligamentum flavum, epidural space, and spinal cord. The classification models provided an average classification accuracy of 96.65%. During puncture, it is important to maintain a safe distance between the needle tip and the dura mater. Regression models were developed to estimate that distance based on the OCT imaging data. Based on the Inception architecture, models achieved a MAPE of 3.05%+0.55%. Overall, the results validated the technical feasibility of using this imaging strategy to automatically recognize different tissue structures and measure the distances ahead of the needle tip during the epidural needle placement. While OCT is discussed, PS-OCT may also be used. PS-OCT may provide more contrast.

### A. Introduction

Epidural anesthesia has become a well-established anesthetic method widely used in painless delivery, thoracic surgeries, orthopedic surgeries, organ transplantation surgeries, abdominal surgeries, and chronic pain relief. Epidural anesthesia uses a needle to inject the anesthetic medications into the epidural space, which averages 1-6 mm in width and several centimeters in depth behind the skin layer. During the placement of the epidural needle, the epidural needle penetrates subcutaneous fat, supraspinous ligament, interspinous ligament, and ligamentum flavum before reaching the epidural space between the flavum and dura mater to inject the medications. Therefore, accurate positioning of the needle in the epidural space is critical for safe and effective epidural anesthesia.

Inadvertent penetration and damage to neurovascular structures leads to several complications, such as headache, transient paresthesia, and severe epidural hematomas. Puncturing the dura will cause excessive loss of cerebrospinal fluid and can damage nerves in the spinal cord. It has been reported that more than 6% of patients have abnormal feelings during the placement of needle, and this has been proved to be a risk factor of persistent paresthesia. PDPH is one of the most common complications in epidural anesthesia. It occurs in over 50% of the accidental dural puncture cases. Some researchers reported the PDPH incidence rate for females was two to three times greater than men, and pregnancy could further increase the possibility of PDPH. Besides PDPH, more serious consequences such as spinal cord damage, paralysis, epidural hematoma, and the development of an abscess might occur due to inaccurate puncture. Moreover, the neurologic injury caused by inadvertent puncture can lead to other symptoms like fever or photophobia.

In current clinical practice, accurate placement of the needle relies on the experience of the anesthesiologist. The most common method of detecting arrival of the needle in the epidural space is based on the LOR. To test the LOR, the anesthesiologist keeps pressing on the plunger of a syringe filled with saline or air while inserting the epidural needle. When the needle tip passes through the ligamentum flavum and arrives at the epidural space, there is a sudden decrease of the resistance that can be sensed by anesthesiologists. Nevertheless, this method has been proved to be inaccurate in predicting needle location, and actual needle insertion could be further inside the body than expected. Up to 10% of patients undergoing epidural anesthesia are not provided with adequate analgesia by using LOR. And the LOR technique can fail in up to 53% of the attempts without image guidance in more challenging procedures such as cervical epidural injections. Moreover, complications such as pneumocephalus, nerve root compression, subcutaneous emphysema, and venous air embolism have proved to be related to the air or liquid injection while using the LOR technique. To improve the success rate of epidural puncture and decrease the number of puncture attempts, there is a strong demand for an effective imaging technique to guide the epidural needle insertion.

Currently, imaging modalities, such as ultrasound and fluoroscopy, have been utilized during the needle access. However, the complex and articulated encasement of bones allows only a narrow acoustic window for the ultrasound beam. Fluoroscopy does not have soft tissue contrast and, thus, cannot differentiate critical soft tissues, such as blood vessels and nerve roots, that need to be avoided during the needle insertion. Moreover, the limited resolution and contrast in fluoroscopy make it difficult to distinguish different tissue layers in front of the needle tip, especially for cervical and thoracic epidural anesthesia where the epidural space is as narrow as 1-4 mm. To improve the needle placement accuracy, novel optical imaging systems have been designed and tested. A portable optical epidural needle system based on fiberoptic bundle was designed to identify the epidural space, but there are some limitations for the optical signal interpretation and needle trajectory identification due to the uncertain direction of the needle bevel or the surrounding fluid. Additionally, optical spectral analysis has been utilized for tissue differentiation during epidural space identification. However, the accuracy of measured spectral results can be compromised by the surrounding tissues and the diffused blood during the puncture.

(OCT is a non-invasive imaging modality that can visualize the cross-sections of tissue samples. At 10-100 times higher resolution (˜10 μm) than ultrasound and fluoroscopy, OCT can improve the efficacy of tissue imaging. OCT has been integrated with fiber-optic catheters and endoscopes for numerous internal imaging applications. Fiber-optic based OCT probe systems have been proposed in epidural anesthesia needle guidance and provided promising results in identifying epidural space in pig models. A forward-imaging endoscopic OCT needle device for real-time epidural anesthesia placement guidance was reported with demonstrated feasibility in piglets in vivo. By fitting the OCT needle inside the hollow bore of the epidural needle, no additional invasiveness is introduced from the OCT endoscope. The high scanning speed of the OCT system allows real-time imaging of the tissue OCT images in front of the needle. The tissues in front of the needle tip can be recognized based on the distinct OCT imaging features of the different tissues.

CNNs have been widely used for classification of medical images and have been applied to OCT images in macular, retina, and esophageal research for automatic tissue segmentation. To help improve the efficiency of tissue recognition, CNNs are used to classify and recognize different epidural tissue types automatically. Specifically, a computer-aided diagnosis system based on CNN is developed to automatically locate the epidural needle tip based on the forward-view OCT images. This is the first attempt to combine a forward-view OCT system with CNNs for guiding the epidural anesthesia procedure. Five epidural layers, including fat, interspinous ligament, ligamentum flavum, epidural space, and spinal cord, were imaged to train and test the CNN classifiers based on Inception, Residual Network 50 (ResNet50), and Xception. After the needle tip arrives in the epidural space, the OCT images can then be used to estimate the distance of the needle tip from the dura mater to avoid spinal cord damage. Regression models were trained and tested based on Inception, ResNet50, and Xception using OCT images with manually-labeled distances. The Inception model achieved the best performance with a MAPE of 3.05%+0.55%. These results demonstrated the feasibility of this imaging strategy for guiding the epidural anesthesia needle placement.

### B. OCT Images of Five Epidural Layer Categories

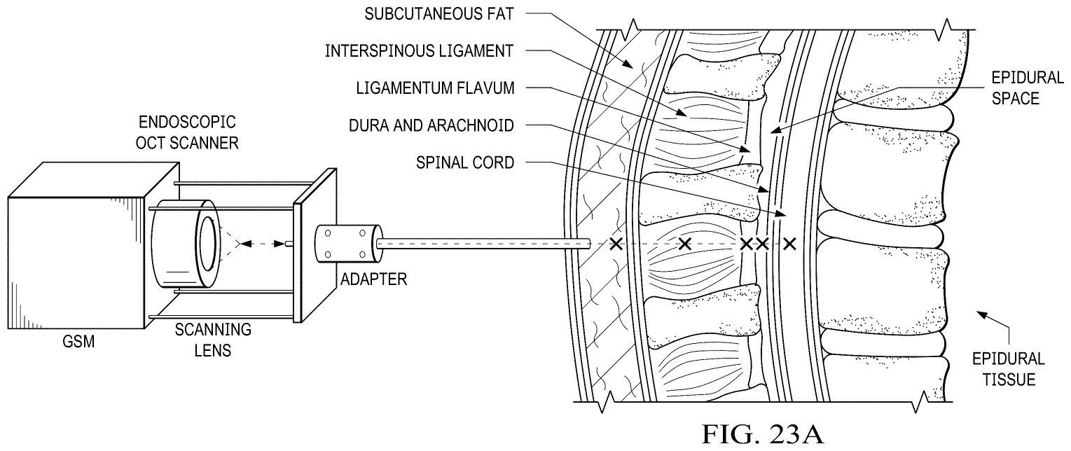

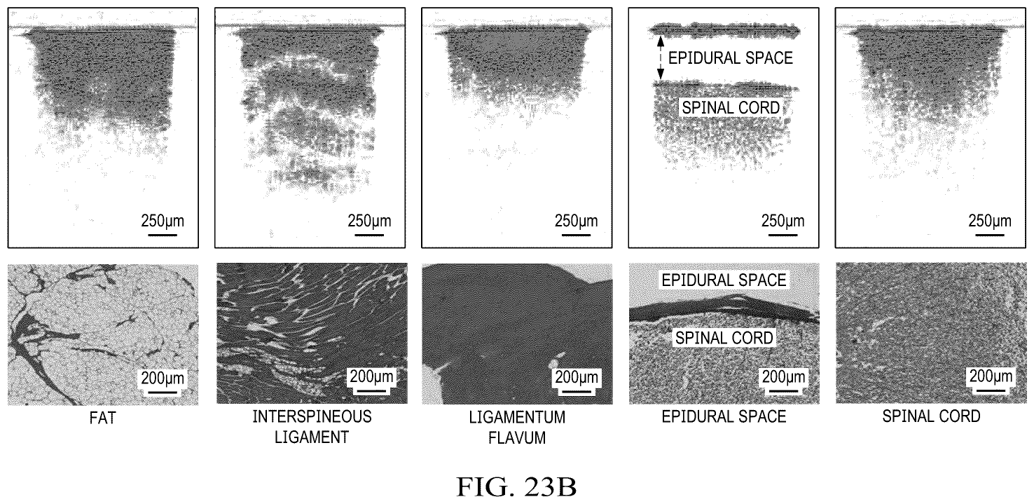

FIG. 23A is a schematic diagram of the experiment using the endoscopic OCT system. FIG. 23B shows cross-sectional 2D OCT image examples of fat, interspinous ligament, ligamentum flavum, epidural space, and spinal cord. Because of the gap between needle tip and dura mater, epidural space was the simplest to be recognized. Among the other four tissues, interspinous ligament showed the most obvious imaging features, including the maximum penetration depth and the clear transverse stripes due to the thick fiber structure. Compared to other tissue types, ligamentum flavum showed higher imaging brightness close to the surface and the shallowest imaging depth. Imaging depths of fat and spinal cord were similar, but the imaging intensity of fat was not as evenly distributed as spinal cord. The corresponding histology results were also included in FIG. 23B. These tissues presented different cellular structures and distributions and correlated well with their OCT results except for fat. The fat tissue was featured with pockets of adipocytes in the histology, while this feature was not clear in the OCT results. This may be caused by the tissue compression applied to mimic the clinical insertion scenario.

### C. Multi-Class Classification of OCT Images by Tissue Layers Using Sequential Binary Method

OCT images of the five tissue layers were classified using CNN models based on three architectures, including ResNet50, Xception, and Inception. However, the overall accuracies of the multi-class classification models based on Inception reached ˜66%. Although this was significantly higher than the accuracy of 20% by random guessing, further improvement was needed for clinical use.

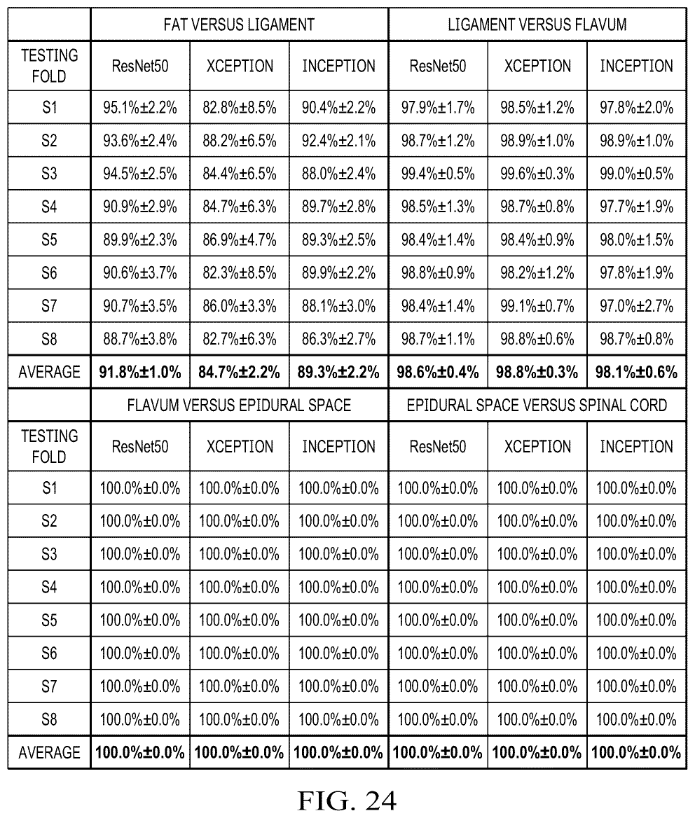

Since the multi-class classification results were not satisfactory, sequential binary methods are used to improve the classification accuracies. During the needle placement, the needle was inserted through fat, interspinous ligament, and ligamentum flavum until reaching the epidural space. Continuing the needle insertion beyond the epidural space can puncture the dura and damage the spinal cord. The classification process was thus divided into a sequential process of four binary classifications: (1) fat versus interspinous ligament; (2) interspinous ligament versus ligamentum flavum; (3) ligamentum flavum versus epidural space; and (4) epidural space versus spinal cord. A FIG. 24 is a table of average accuracies and standard error based on the practical tissue layer sequence during puncture for cross-validation.

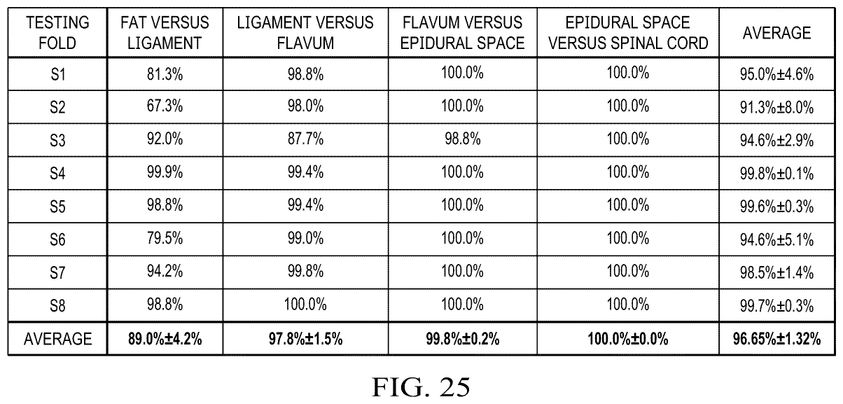

Overall, ResNet50 showed the best prediction results. FIG. 25 is a table further showing the test accuracy of the best-performing model (ResNet50) in each of the 8 testing folds. Almost all the results were over 90%. There was substantial variability in the test accuracy among different subjects, especially for the prediction accuracy of “Fat vs Interspinous Ligament.” While three subjects had test accuracies higher than 98.8%, the subjects in the S2 fold had the lowest test accuracy of 67.3%. This may be due to the tissue variability among different backbone samples and the different tissue compression during imaging, especially considering fat is subject to tissue compression. The areas AUC differed among different samples.

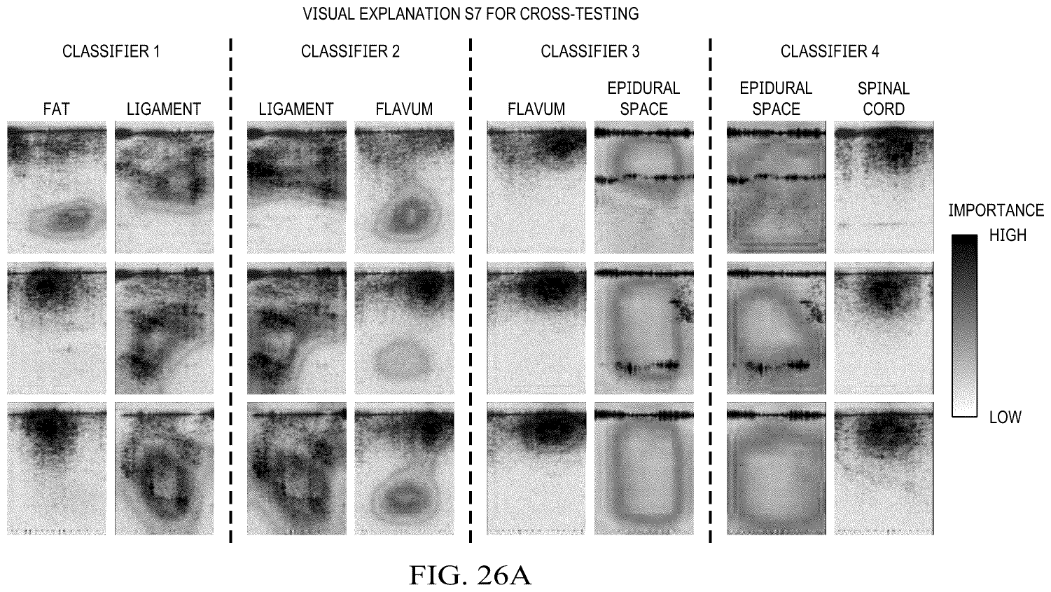

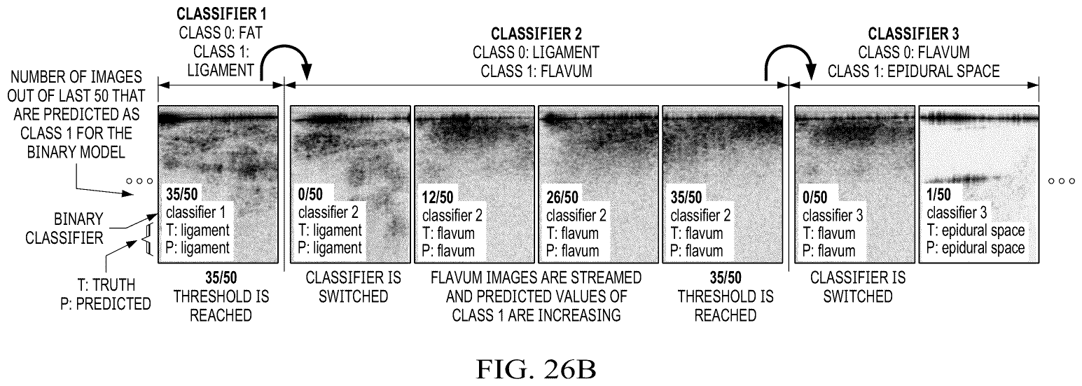

FIG. 26A shows class activation heatmaps of ResNet50 models for representative images to show the salient features used for classification. Each binary classification model paid attention to different regions of the images. For example, the black empty space was important for the models to recognize the epidural space images. A video stream of the OCT images was used to demonstrate the sequential binary models. The number of images was 100, 700, 100, 100, and 150 for fat, interspinous ligament, ligamentum flavum, epidural space, and spinal cord, respectively, which was proportional to the width of these tissue layers. After the binary classifier of fat versus ligament detects 35 interspinous ligament images in the last 50 images, the needle was considered to be in the interspinous ligament and the next binary classifier of interspinous ligament versus ligamentum flavum was activated to detect the upcoming ligamentum flavum. This simple logic was used to switch all the subsequent classifiers. FIG. 26B shows some images from a video that can be found in the Github repository. The images include scenes from the video showing the switch from classifier 2 to classifier 3 and its arrival to epidural space. Each image showed three important pieces of information. First, the images showed the proportion of the last 50 images that were predicted to belong to class 1. Class I was interspinous ligament in the first classifier and was ligamentum flavum in the second classifier. Initially, when the number of images was less than 50, the denominator shows the total number of images. Additionally, the tissue recognition results were color-coded for the needle insertion like traffic lights for vehicle navigation. It changed from green to yellow at 26 and from yellow to red at 35. The second information was the current classifier. The last information was the truth and predicted label. The switch of binary classifier occurred when the number of images predicted as class 1 reached 35. The fraction did not appear anymore when the last classifier was reached.

### D. Estimation of the Distance Between the Needle Tip and Dura Mater by Regression

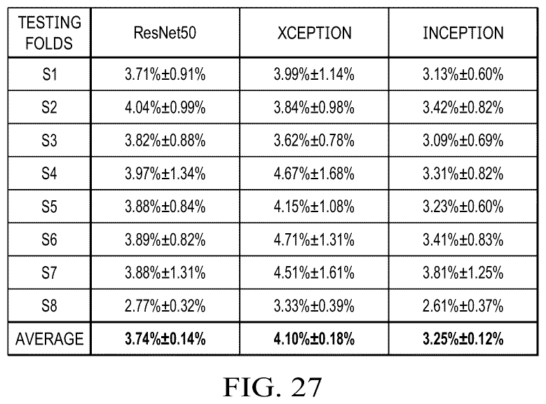

Inception, ResNet50, and Xception were compared for the regression task of estimating the distance of the needle tip to the dura mater. FIG. 27 is a table showing the mean and standard error of the cross-validation MAPE for ResNet50, Xception, and Inception in all testing folds. In every fold, the Inception model outperformed the ResNet50 and Xception models as indicated by the lowest MAPE.

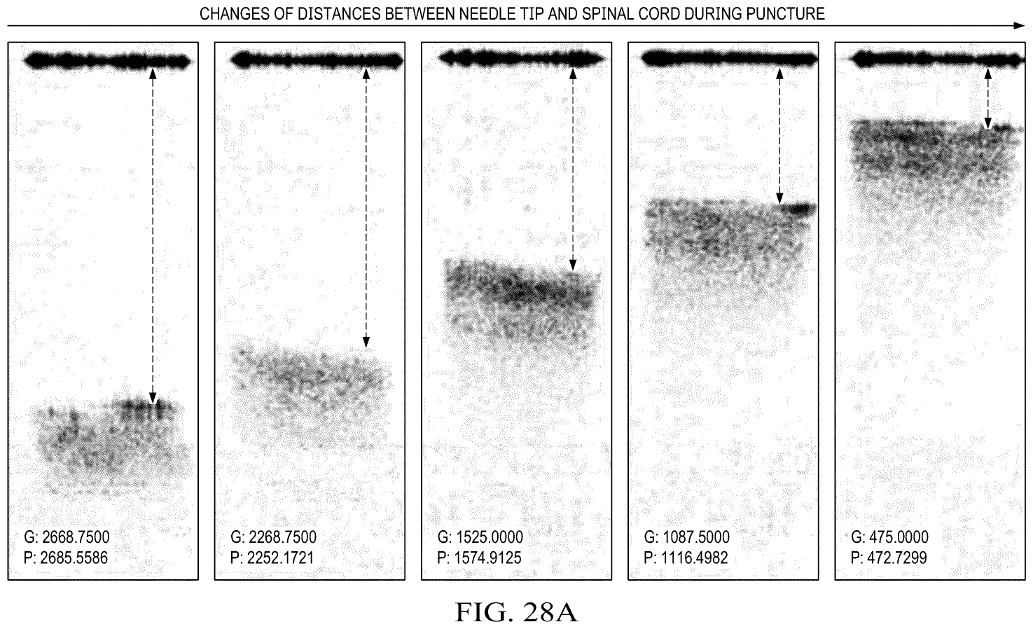

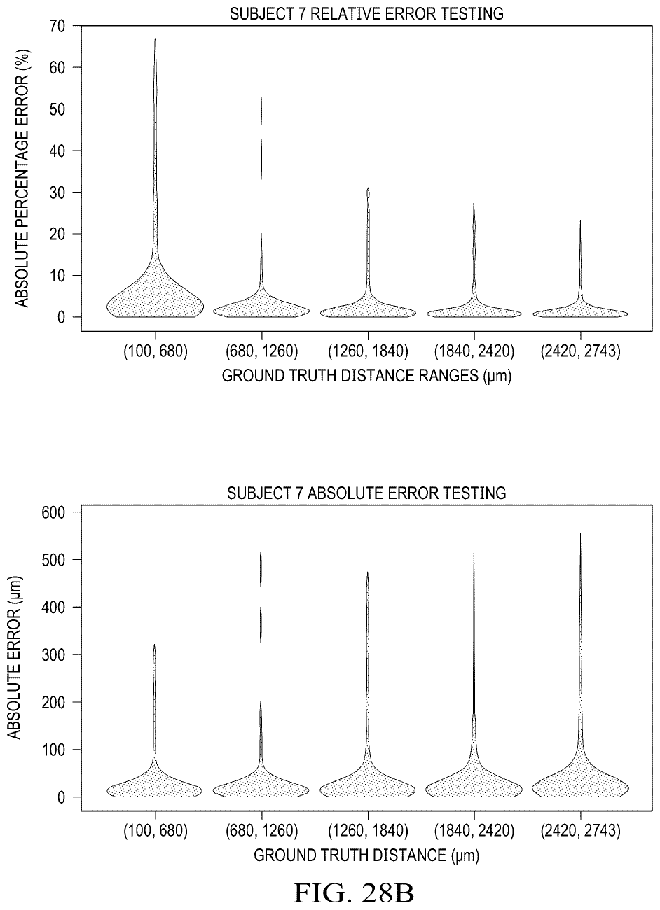

In each testing rotation, a new Inception model was trained using all the images in the seven cross-validation folds and then evaluated on the unseen testing images in the one testing fold. FIG. 28A shows examples of OCT images with different distances between needle tip and tissue. A model was trained on 21,000 images belonging to subjects 1, 2, 3, 4, 5, 6, and 8, and tested on 3,000 images belonging to subject 7. FIG. 28B shows violin plots of the distribution of the errors from the Inception model during the seventh testing fold (i.e., testing images belong to subject 7). The MAPE on this testing set was 3.626%, and the MAE was 34.093 μm. From the testing results on the Inception architecture, it was evident that the regression model can accurately estimate the distance to the dura mater in most of the OCT images.

### E. Discussion

The study validated the endoscopic OCT system for epidural anesthesia surgery guidance. The OCT endoscope can provide 10-100 times higher resolution than other medical imaging modalities. Moreover, this proposed endoscopic OCT system is compatible with the clinical-used epidural guiding methods (e.g., ultrasound, fluoroscopy, and CT), and will complement these macroscopic methods by providing the detailed images in front of the epidural needle.

Five different tissue layers, including fat, interspinous ligament, ligamentum flavum, epidural space, and spinal cord, were imaged. To assist the OCT image interpretation, a deep learning-based CAD platform was developed to automatically differentiate the tissue layers at the epidural needle tip and predict the distance from the needle tip to dura mater.

Three CNN architectures, including ResNet50, Xception, and Inception, were tested for image classification and distance regression. The best classification accuracy of the five tissue layers were 60-65% from a multi-class Inception classifier. The main challenge was the differentiation between fat and spinal cord because they had similar feature in OCT images. Based on the needle puncture sequence, the overall classification was divided into four sequential binary classifications: fat versus interspinous ligament, interspinous ligament versus ligamentum flavum, ligamentum flavum versus epidural space, and epidural space versus spinal cord. The overall prediction accuracies of all four classifications reached to more than 90%. ResNet50 presented the best overall performance compared to Xception and Inception. Due to the unique features of the epidural space in OCT images, it was possible to achieve >99% precision of when the needle arrived the epidural space. Classifying epidural space versus ligamentum flavum and epidural space versus spinal cord had accuracies of ˜99.8% and 100%, respectively. This will allow accurate detection of the epidural space for injection of the anesthetic during epidural anesthesia. The sequential transition from one binary classifier to the next was controlled accurately using a simple logic, which was demonstrated in a video simulating the insertion of a needle through the five tissue layers. In the future, this can be improved by combining CNNs with recurrent neural networks to handle the temporal dimension of video streaming data. Additionally, a CNN regression model was developed to estimate the needle distance to the dura mater upon entry of the epidural space. For the regression task, Inception provided better performance compared to Xception and ResNet50. The mean relative error was 3.05%, thus demonstrating the ability to track the accurate location of the needle tip in the epidural space.

CNNs have shown to be a valuable tool in biomedical imaging. Manually configuring CNN architectures for an imaging modality can be a tedious trial-and-error process. ResNet, Inception, and Xception are commonly used architectures for general image classification tasks. The architectures can be adapted for both classification and regression tasks in biomedical imaging applications. The best performance was obtained by ResNet50 for the binary classifications and by Inception for the distance regression.

The nested cross-validation and testing procedure was computationally expensive, but it provided the uncertainty quantification of the test performance across subjects. The wall-clock times for training the binary classification models on NVIDIA Volta GPUs were ˜11 minutes per validation fold for ResNet50, ˜32 minutes per validation fold for Xception, and ˜11 minutes per validation fold for Inception. The wall-clock times for training the regression models on NVIDIA RTX 3090 GPUs were ˜50 minutes per validation fold for ResNet50, ˜145 minutes per validation fold for Xception, and ˜36 minutes per validation fold for Inception. The inferencing for the binary classifications on NVIDIA Volta GPUs took 13 ms per image on average. The inferencing for the distance regression on NVIDIA RTX 3090 GPUs took 2.1 ms per image on average. In the future, the inferencing by these large CNN models can be further accelerated by weight pruning and knowledge distillation.

The GRIN lens can be used with a suitable diameter for a practical 16-gauge Tuohy needle used in epidural anesthesia. Furthermore, the size of the OCT scanner can be miniaturized to make the system more portable and convenient for anesthesiologists to use in clinical applications. Finally, the performance of the endoscopic OCT system can be tested together with the deep learning-based CAD platform in the in-vivo pig experiments. Differences of OCT images from in-vivo and ex-vivo conditions may deteriorate the in-vivo testing results. In that case, the model can be re-trained using in vivo pig data. Additionally, during the in-vivo experiments, there will be blood vessels surrounding the spinal cord. To address this, a Doppler OCT method can be used for the blood vessel detection to avoid the rupture of blood vessels during epidural needle insertion.

### F. Experimental Setup

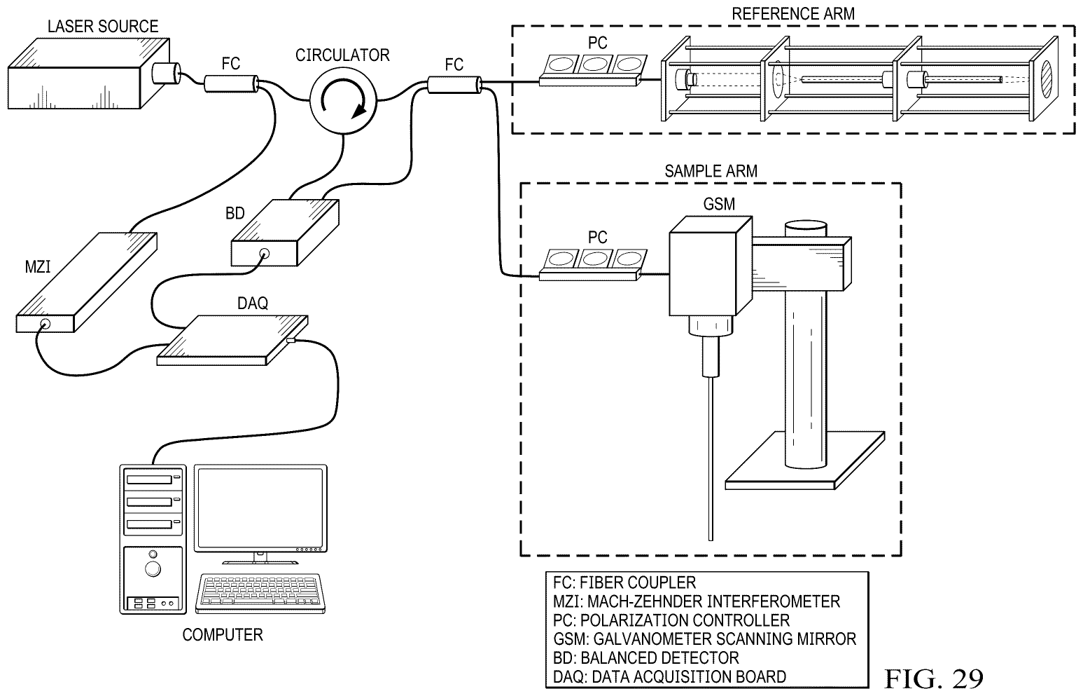

FIG. 29 is a schematic diagram of the forward-view OCT endoscope. Its working principle was based on a Michaelson interferometer with a reference arm and a sample arm. The endoscopic system was built on an SS-OCT. The light source was a wavelength-swept laser with a 1,300 nm central wavelength and a 100 nm bandwidth. The laser had the maximum scanning speed at 200 kHz A-Scan rate. The light from the laser was first unevenly split by an FC. 97% power was split into the circulator and transmitted into the interferometer, and the other 3% was input to the MZI, which provided the triggering signal for data sampling. The 97% power was further split by another 50:50 FC to the reference arm and the sample arm. The reflected signal from the reference arm and the backscattered signal from the sample arm interfered with each other and were collected by a BD for noise reduction. The signal was then sent to a DAQ board and computer for post-processing based on the Fourier transform. While imaging the samples in the air, the axial resolution reached to 10.6 μm and the lateral resolution was 20 μm.

To achieve the endoscopic imaging, a GRIN rod lens was added in the sample arm. It was fixed in front of the scanning lens of the GSM. The GRIN lens had a total length of 138 mm, an inner diameter of 1.3 mm, and a view angle of 11.0°. It was protected by a thin-wall steel tubing. For dispersion compensation, a second set of identical GRIN lens was stabilized in front of the reflector (mirror) of the reference arm. In addition, two PCs were placed in each arm to reduce the noise level.

The GRIN lens utilized in the sample arm was assembled in front of the OCT scanning lens of the GSM. To decrease the reflection from the proximal end surface of the GRIN lens that significantly degraded the imaging quality, the proximal surface of the GRIN lens was aligned ˜1.5 mm off the focus of the scanning lens. The GRIN lens can relay the images from the distal end to its proximal surface. In the sample arm, the proximal GRIN lens surface was adjusted close to the focus point of the objective after the OCT scanner. Thus, the spatial information from the distal surface (tissue sample) of the GRIN lens transmitted to the proximal surface was further collected by the OCT scanner. Therefore, OCT images of the epidural tissues in front of the GRIN lens can be successfully obtained. The endoscopic system provided an ˜1.25 mm FOV with a sensitivity of 92 dB.

### G. Data Acquisition

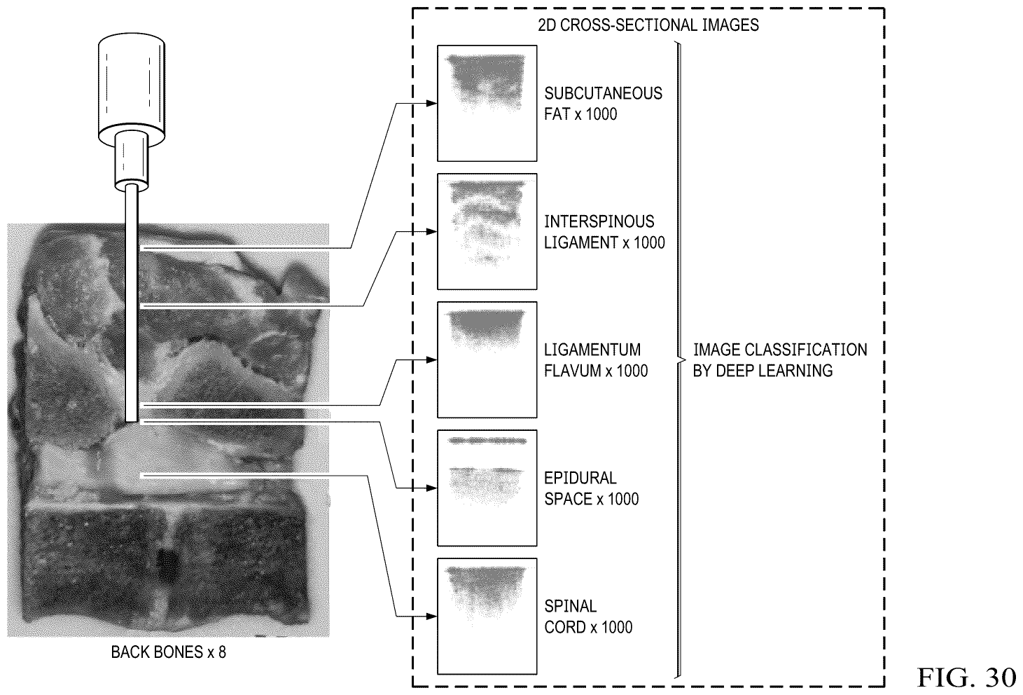

FIG. 30 is a diagram showing the data acquisition process. Backbones from eight pigs were acquired from local slaughterhouses and cut at the middle before imaging to expose different tissue layers. From the cross-section of the sample, different tissue types could be clearly distinguished through the tissue anatomic features and their positions as shown. To further limit the number of misclassified results, two lab members confirmed the tissue types before imaging started. Five tissue layers, including fat, interspinous ligament, ligamentum flavum, epidural space, and spinal cord, can be distinguished from their anatomic appearance. The OCT needle was placed against these confirmed tissue layers to obtain their OCT structural images. Following the practice of epidural needle placement, the puncturing process was mimicked by inserting the OCT endoscope through fat, interspinous ligament, ligamentum flavum, and epidural space of the sample. Since the targeted position of the anesthetic injection is the epidural space with a width ˜1-6 mm, OCT images of epidural space were obtained by positioning the needle tip in front of the spinal cord at different distances. To mimic the condition of accidental puncture into the spinal cord, OCT images were taken while inserting the endoscope into the spinal cord. Some force was applied during imaging the four tissue types (fat, interspinous ligament, ligamentum flavum, and spinal cord) to generate compression to better represent the actual in-vivo clinical situation.

For each backbone sample, 1,000 cross-sectional OCT images were obtained from each tissue layer. To decrease noise and increase the deep-learning processing speed, the original images were further cropped to smaller sizes that only contained the effective tissue information. Images were cropped to 181×241 pixels for the tissue classification.

At the end of imaging, tissues of fat, interspinous ligament, ligamentum flavum, and spinal cord with dura mater of the porcine backbones were excised and processed for histology following the same orientation of OCT endoscope imaging to compare with corresponding OCT results. The tissues were fixed with 10% formalin, embedded in paraffin, sectioned to 4 μm thick, and stained with hematoxylin and cosin for histological analysis.

### H. CNNs

(CNNs were used to classify OCT images by epidural layers. Three CNN architectures, including ResNet50, Inception, and Xception, were imported from the Keras library. The output layer of the models was a dense layer that represented the number of categories. The images were centered by subtracting training mean pixel value. The SGD with Nesterov momentum optimizer was used with a learning rate of 0.01, a momentum of 0.9, and a decay of 0.01. The batch size was 32. Early stopping was used with a patience of 10. The loss function used was sparse categorical cross-entropy.

Nested cross-validation and testing were used for model selection and benchmarking as described previously. This evaluation strategy provided an unbiased estimation of model performance with uncertainty quantification using two nested loops for cross-validation and cross-testing. Images were acquired from eight subjects in this dataset. The images were divided to 8 folds by subjects to account for the subject-to-subject variability. An eight-fold cross-testing loop was performed by rotating through every subject for testing and using the remaining seven subjects (7,000 images) for cross-validation. In the cross-validation, six subjects were used for training and one subject for validation in each rotation. The 7-fold cross-validation loop was used to compare the performance of three architecture models: ResNet50, Xception and Inception. The model with the best cross-validation performance was automatically selected for performance benchmarking in the corresponding testing fold. The performance of this overall procedure was evaluated by aggregating the testing performance from all 8 testing folds.

The classification accuracy of the models was computed using equation (1)

ROC curves were used to visualize the relationship between sensitivity and specificity. The AUC of ROC was also used to assess the overall performance of the models.

### I. Epidural Distance Prediction Using Deep Learning

OCT images of epidural space were obtained at a range of distances between approximately 0.2 mm and 2.5 mm from the needle tip to the spinal cord surface (dura mater). A total of 24,000 images from eight subjects were used for this task. For each image taken in the epidural space for the distance estimation task, the distance in micrometers from the epidural needle to the dura mater was manually calculated and labeled. This distance label served as the ground truth for computing the loss during the training process in the regression model. All images were of 241×681 pixels with a pixel size of 6.25 μm. The pixel values for each image were scaled in the range of 0-255.

The regression model was developed to estimate the distance from the epidural needle to the dura upon entry into the epidural space automatically. Three architectures, including ResNet50, Inception, and Xception, were compared using nested cross-validation and testing as described above. The final output layer consisted of a single neuron with an identity activation function for regression on the continuous distance values. The SGD algorithm with Nesterov momentum optimization was used with a learning rate of 0.01, momentum of 0.9, and decay rate of 0.01. Training took place with a batch size of 32 over 20 epochs. The (MAPE and MAE were the metrics used to evaluate the regression performance due to their intuitive interpretability in relation to the relative error. The MAPE and MAE performance metrics are defined in equations (2) and (3), respectively.

### J. Embodiments

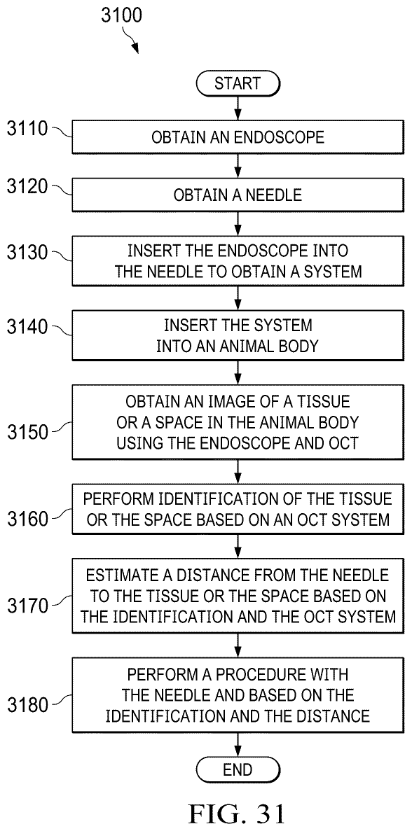

FIG. 31 is a flowchart illustrating a method 3100 for endoscopy based on OCT and CNNs according to one non-limiting embodiment of the present disclosure. At step 3110, an endoscope is obtained. At step 3120, a needle is obtained. At step 3130, the endoscope is inserted into the needle to obtain a system. At step 3140, the system is inserted into an animal body. At step 3150, an image of a tissue or a space in the animal body is obtained using the endoscope and OCT. At step 3160, identification of the tissue or the space is performed based on an OCT system. At step 3170, a distance from the needle to the tissue or the space is estimated based on the identification and the OCT system. Finally, at step 3180, a procedure is performed with the needle and based on the identification and the distance.

The method 3100 may comprise additional embodiments. For instance, The OCT system is based on OCT images and CNNs. The CNNs comprise a first CNN associated with the identification and a second CNN associated with the distance. The first CNN comprises a classification model. The second CNN comprises a regression model. The needle is a Veress needle. The needle is a Tuohy needle. The tissue is subcutaneous fat, a muscle, or an intestine. The tissue is a backbone tissue. The backbone tissue is fat, an interspinous ligament, a ligamentum flavum, or a spinal cord. The space is an abdominal space. The space is an epidural space. The method further comprises further performing the procedure independent of LOR. The procedure is laparoscopy. The procedure is epidural anesthesia.

While several embodiments have been provided in the present disclosure, it may be understood that the disclosed systems and methods might be embodied in many other specific forms without departing from the spirit or scope of the present disclosure. The present examples are to be considered as illustrative and not restrictive, and the intention is not to be limited to the details given herein. For example, the various elements or components may be combined or integrated in another system or certain features may be omitted, or not implemented.

In addition, techniques, systems, subsystems, and methods described and illustrated in the various embodiments as discrete or separate may be combined or integrated with other systems, components, techniques, or methods without departing from the scope of the present disclosure. Other items shown or discussed as coupled may be directly coupled or may be indirectly coupled or communicating through some interface, device, or intermediate component whether electrically, mechanically, or otherwise. Other examples of changes, substitutions, and alterations are ascertainable by one skilled in the art and may be made without departing from the spirit and scope disclosed herein.

What is claimed is:

1. A method comprising:
obtaining an endoscope;
obtaining a needle;
inserting the endoscope into the needle to obtain a system;
inserting the system into an animal body;
obtaining an image of a tissue or a space in the animal body using the endoscope and optical coherence tomography (OCT);
performing identification of the tissue or the space based on an OCT system;
estimating a distance from the needle to the tissue or the space based on the identification and the OCT system; and
performing a procedure with the needle and based on the identification and the distance.

2. The method of claim 1, wherein the OCT system is based on OCT images and convolutional neural networks (CNNs).

3. The method of claim 2, wherein the CNNs comprise a first CNN associated with the identification and a second CNN associated with the distance, wherein the first CNN comprises a classification model, and wherein the second CNN comprises a regression model.

4. The method of claim 1, wherein the needle is a Veress needle.

5. The method of claim 1, wherein the needle is a Tuohy needle.

6. The method of claim 1, wherein the tissue is subcutaneous fat, a muscle, or an intestine.

7. The method of claim 1, wherein the tissue is a backbone tissue, and wherein the backbone tissue is fat, an interspinous ligament, a ligamentum flavum, or a spinal cord.

8. The method of claim 1, wherein the space is an abdominal space.

9. The method of claim 1, wherein the space is an epidural space.

10. The method of claim 1, further comprising further performing the procedure independent of loss of resistance (LOR).

11. The method of claim 1, wherein the procedure is laparoscopy.

12. The method of claim 1, wherein the procedure is epidural anesthesia.

13. A system comprising:
a needle configured to insert into an animal body;
an endoscope configured to:
insert into the needle; and
obtain an image of a tissue or a space in an animal body using optical coherence tomography (OCT); and
a processor configured to:
perform identification of the tissue or the space based on an OCT system; and
estimate a distance from the needle to the tissue or the space based on the identification and the OCT system.

14. The system of claim 13, wherein the OCT system is based on OCT images and convolutional neural networks (CNNs).

15. The system of claim 14, wherein the CNNs comprise a first CNN associated with the identification and a second CNN associated with the distance, wherein the first CNN comprises a classification model, and wherein the second CNN comprises a regression model.

16. The system of claim 13, wherein the needle is a Veress needle or a Tuohy needle.

17. The system of claim 13, wherein the tissue is subcutaneous fat, a muscle, or an intestine.

18. The system of claim 13, wherein the tissue is a backbone tissue, and wherein the backbone tissue is fat, an interspinous ligament, a ligamentum flavum, or a spinal cord.

19. The system of claim 13, wherein the space is an abdominal space or an epidural space.

20. The system of claim 13, wherein the needle is further configured to perform a procedure based on the identification, based on the distance, and independent of loss of resistance (LOR), and wherein the procedure is laparoscopy or epidural anesthesia.
